(19) United States
(12) Patent Application Publication
(10) Pub. No.: US 2026/0295041 A1
(43) Pub. Date: Oct. 1, 2026

(54) Use of Amivantamab to Treat Head and Neck Cancer

(71) Applicant: Janssen Biotech, Inc, Horsham, PA (US)

(72) Inventors: Mahadi Baig, Brookfield, WI (US); Emrullah Yilmaz, Beachwood, OH (US); Chunying Gao, Malvern, PA (US)

(21) Appl. No.: 19/542,806

(22) Filed: Feb. 18, 2026

Related U.S. Application Data
(60) Provisional application No. 63/759,852, filed on Feb. 18, 2025.
(60) Provisional application No. 63/855,717, filed on Aug. 1, 2025.

(51) Int. Cl. A61K 39/395; A61K 31/282; A61K 39/00; A61P 35/00; C07K 16/28

(52) U.S. Cl. CPC A61K 39/39558; A61K 31/282; A61P 35/00; C07K 16/2827; C07K 16/2863; A61K 2039/507; A61K 2039/54; A61K 2039/545; C07K 2317/31

(57) ABSTRACT

The present invention relates to methods of treating head and neck squamous cell carcinoma (HNSCC), such as metastatic or advanced HNSCC, in a subject in need thereof, comprising administering a therapeutically effective amount of an antibody (e.g., a bispecific antibody) to the subject, wherein the antibody specifically binds epidermal growth factor receptor (EGFR) and hepatocyte growth factor receptor (c-Met), in addition to a PD-(L)1 axis inhibitor and a platinum-based chemotherapy agent.

### CROSS-REFERENCE TO RELATED APPLICATIONS

This application claims the benefit of U.S. Provisional Application No. 63/759,852, filed on 18 Feb. 2025 and U.S. Provisional Application No. 63/855,717, filed on 1 Aug. 2025, each of which is incorporated herein by reference in their entirety.

### SEQUENCE LISTING

The instant application contains a Sequence Listing, which has been submitted electronically in XML format and is hereby incorporated by reference in its entirety. Said XML copy, created on Feb. 17, 2026, is named JBI6981USNP1_SL.xml, and is 45,376 bytes in size.

### FIELD OF THE INVENTION

The present invention relates to methods of treating head and neck squamous cell carcinoma (HNSCC), such as recurrent or metastatic HNSCC, in a subject in need thereof, comprising administering a therapeutically effective amount of an antibody (e.g., a bispecific antibody) to the subject, wherein the antibody specifically binds epidermal growth factor receptor (EGFR) and hepatocyte growth factor receptor (c-Met), in addition to a PD-(L)1 axis inhibitor and a platinum-based chemotherapy agent.

### BACKGROUND

HNSCC represents a significant global health concern and is the sixth most common cancer worldwide, with 890,000 new cases and 450,000 deaths in 2018 (Johnson 2020). In the United States, approximately 66,000 cases will be diagnosed annually, and 15,000 deaths will occur (Siegel 2022). There are multiple risk factors for HNSCC, including tobacco use, alcohol consumption, and HPV infection, identified by positive p16 status, which plays a significant role in the development of squamous cell carcinomas of the oropharynx (Machiels 2020). Patients with p16-positive oropharyngeal cancer have better sensitivity to chemotherapy and radiotherapy regimens compared to patients with p16-negative disease (Machiels 2020; Perri 2020); however, studies also suggest that patients with p16-positive disease respond more poorly to anti-EGFR therapy in comparison to patients with p16-negative disease (Taberna 2019).

Around 50% of the previously treated population will experience local recurrence or distant metastasis within 5 years (Machiels 2020). The majority of patients with disease recurrence are not candidates for curative intent therapy, so recurrent and/or metastatic (R/M) disease is generally treated with systemic therapy with palliative intent, with variable therapeutic options. In patients with platinum pretreated R/M HNSCC, the ORR is approximately 10% for cetuximab monotherapy (Vermorken 2007; Cohen 2006) and 14% for paclitaxel monotherapy (Soulières 2017). For patients with treatment-naïve R/M HNSCC with PD-L1 CPS≥1, pembrolizumab monotherapy is associated with an ORR of 19% (Burtness 2019). These response rates highlight the need for novel therapies to be explored in this population of high unmet clinical need.

The current Standard of Care (SoC) first-line therapy for patients with R/M HNSCC has been established by the KEYNOTE-048 study (Burtness 2019), which demonstrated that pembrolizumab in addition to chemotherapy significantly improved OS (median OS: 13 months) compared to the EXTREME regimen comprised of cetuximab, platinum-based chemotherapy, and 5-fluorouracil (median OS: 10.7 months). In comparison to the EXTREME regimen, pembrolizumab monotherapy also demonstrated improved OS in patients with PD-L1 positive HNSCC (median OS: 12.3 months versus 10.3 months in patients with a PD-L1 CPS≥1) (Burtness 2019). Pembrolizumab monotherapy also demonstrated an improved safety profile compared with the EXTREME regimen, with the exception of endocrine disorders (Burtness 2019). For patients with a PD-L1 CPS≥1, pembrolizumab monotherapy is a therapeutic consideration as front-line therapy (Machiels 2020; NCCN Clinical Practice Guidelines 2022). The toxicities associated with pembrolizumab in addition to chemotherapy are substantial, therefore many patients will receive pembrolizumab monotherapy as front-line therapy. There remains a need to improve overall response rates and outcomes while maintaining the acceptable safety profile demonstrated with pembrolizumab monotherapy.

### SUMMARY

There is a need for improved therapeutics or combinations of therapeutics to develop more effective treatment of head and neck cancer, including head and neck squamous cell carcinoma (HNSCC).

The disclosure generally relates to methods that are useful for treating head and neck cancer (e.g., HNSCC). In certain embodiments, the methods are useful for treating recurrent or metastatic head and neck squamous cell carcinoma (R/M HNSCC).

In one aspect, the disclosure provides a method of treating head and neck cancer in a subject in need thereof, comprising administering to the subject a combination therapy, wherein the combination therapy comprises (1) a therapeutically effective amount of an anti-epidermal growth factor receptor (EGFR)/hepatocyte growth factor receptor (c-Met) antibody; (2) a therapeutically effective amount of pembrolizumab; and (3) a therapeutically effective amount of carboplatin.

In some embodiments, the antibody is a bispecific antibody.

In some embodiments, the antibody comprises:
a first domain that specifically binds EGFR, comprising heavy chain complementarity determining region 1 (HCDR1), HCDR2, HCDR3, light chain complementarity determining region 1 (LCDR1), LCDR2 and LCDR3 amino acid sequences of SEQ ID NOs: 1, 2, 3, 4, 5 and 6, respectively; and a second domain that specifically binds c-Met, comprising HCDR1, HCDR2, HCDR3, LCDR1, LCDR2 and LCDR3 amino acid sequences of SEQ ID NOs: 7, 8, 9, 10, 11 and 12, respectively.

In some embodiments, the first domain comprises a heavy chain variable region (VH) of SEQ ID NO: 13 and a light chain variable region (VL) of SEQ ID NO:14, and the second domain comprises a VH of SEQ ID NO:15 and a VL of SEQ ID NO:16.

In some embodiments, the antibody is of the IgG1 isotype.

In some embodiments, the antibody comprises a first heavy chain (HC1) of SEQ ID NO: 17, a first light chain (LC1) of SEQ ID NO:18, a second heavy chain (HC2) of SEQ ID NO: 19 and a second light chain (LC2) of SEQ ID NO:20.

In some embodiments, the antibody is an isolated bispecific antibody.

In some embodiments, the bispecific antibody is amivantamab.

In some embodiments, the antibody comprises a biantennary glycan structure with a fucose content of about 1% to about 15%. In some embodiments, the antibody comprises a biantennary glycan structure with a fucose content of less than about 20%.

In some embodiments, the antibody is administered at a dose of about 1,600 mg to about 3,360 mg.

In some embodiments, the antibody is administered at a dose of about 1,600 mg, about 2,240 mg, about 2,400 mg, or about 3,360 mg.

In some embodiments, the antibody is administered at a dose of about 1,600 mg.

In some embodiments, the antibody is administered at a dose of about 2,240 mg.

In some embodiments, the antibody is administered at a dose of about 2,400 mg.

In some embodiments, the antibody is administered at a dose of about 3,360 mg.

In some embodiments, the antibody is administered once a week or once every three weeks. In some embodiments, the antibody is administered once weekly for the first 3 weeks and once every 3 weeks thereafter.

In some embodiments, the antibody is administered at a dose of about 1,600 mg (2240 mg, if body weight is equal or larger than 80 kg) on Cycle 1 Day 1 and 2400 mg (3360 mg, if body weight is equal or larger than 80 kg) once every week (Q1W) for the remainder of Cycle 1 (Days 8 and 15), and once every 3 weeks (Q3W) from Cycle 2 onwards, wherein each Cycle is 21 days.

In some embodiments, the antibody is administered at a dose of about 1,600 mg on Cycle 1 Day 1 and about 2400 mg once every week (Q1W) for the remainder of Cycle 1 (Days 8 and 15), and once every 3 weeks (Q3W) from Cycle 2 onwards, wherein each Cycle is 21 days, if body weight is less than 80 kg.

In some embodiments, the antibody is administered at a dose of about 2240 mg on Cycle 1 Day 1 and about 3360 mg once every week (Q1W) for the remainder of Cycle 1 (Days 8 and 15), and once every 3 weeks (Q3W) from Cycle 2 onwards, wherein each Cycle is 21 days, if body weight is equal or larger than 80 kg.

In some embodiments, pembrolizumab is administered at a dose of about 200 mg once every three weeks.

In some embodiments, carboplatin is administered at a dose of about AUC 5 mg/mL·min once every three weeks.

In some embodiments, the head and neck cancer is a head and neck squamous cell carcinoma (HNSCC). In some embodiments, the HNSCC is cancer of oropharynx, oral cavity, hypopharynx, or larynx.

In some embodiments, the HNSCC is HPV unrelated. In some embodiments, the HNSCC is HPV negative. In some embodiments, the HNSCC is HPV positive. In some embodiments, the HNSCC HPV infection status is unknown.

In some embodiments, the subject is treatment naïve. In some embodiments, the subject is treatment naïve in the recurrent or metastatic setting.

In some embodiments, the subject was administered at least one anti-cancer therapy previously.

In some embodiments, the subject is 18 years of age or older.

In one aspect, the disclosure provides a method of treating recurrent or metastatic head and neck squamous cell carcinoma in a subject in need thereof comprising administering a therapeutically effective amount of the anti-EGFR/anti-cMet antibody (e.g., amivantamab), pembrolizumab, and carboplatin, wherein the subject is treatment-naïve in a recurrent/metastatic head and neck cancer setting, wherein the anti-EGFR/anti-cMet antibody is administered in 21-day cycles (i) in an amount of 1,600 mg if the subject has a body weight of less than 80 kg (or an amount of 2,240 mg if the subject has a body weight of ≥80 kg) on Cycle 1 Day 1, and then (ii) in an amount of 2,400 mg if the subject has a body weight of less than 80 kg (or an amount of 3,360 mg if the subject has a body weight of ≥80 kg) once weekly (Q1W) for the remainder of Cycle 1 (Days 8 and 15) and once every 3 weeks (Q3W) from Cycle 2 onwards, and wherein the pembrolizumab is administered at a dose of 200 mg Q3W (on Day 1 of each 21-day cycle) starting in Cycle 1 (e.g., as an intravenous injection), and wherein the carboplatin is administered at a dose of AUC 5 mg/mL·min Q3W (on Day 1 of each 21-day cycle) starting in Cycle 1 (e.g., as an intravenous injection).

In some embodiments, the anti-EGFR/anti-cMet antibody is administered subcutaneously.

In some embodiments, the anti-EGFR/anti-cMet antibody is administered intravenously.

In some embodiments, the method achieves a partial response or complete response in the subject, as defined by investigator assessment using Response Criteria in Solid Tumors (RECIST) version 1.1.

### BRIEF DESCRIPTION OF THE DRAWINGS

FIG. 1 shows best change from baseline in Sum of Diameters (SoD) of target lesions, calculated for each patient.

FIG. 2 shows changes from baseline in SoD of target lesions for each patient.

FIG. 3 shows the base line CT scan for Patient 1.

FIG. 4 shows the 13 week CT scan for Patient 1.

FIG. 5A and FIG. 5B show the base line CT scans for Patient 2.

FIG. 6A and FIG. 6B show the 7 week CT scans for Patient 2

FIG. 7 shows the clinical trial schema.

### DETAILED DESCRIPTION

### Definitions

All publications, including but not limited to patents and patent applications, cited in this specification are herein incorporated by reference as though fully set forth.

It is to be understood that the terminology used herein is for describing particular embodiments only and is not intended to be limiting. Unless defined otherwise, all technical and scientific terms used herein have the same meaning as commonly understood by one of ordinary skill in the art to which the invention pertains.

Although any methods and materials similar or equivalent to those described herein may be used in the practice for testing of the present invention, exemplary materials and methods are described herein. In describing and claiming the present invention, the following terminology will be used.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The conjunctive term “and/or” between multiple recited elements is understood as encompassing both individual and combined options. For instance, where two elements are in conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

The term “antibody” or “antibodies” is meant in a broad sense and includes immunoglobulin molecules including monoclonal antibodies including murine, human, humanized and chimeric monoclonal antibodies, full-length antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity.

“Specific binding” or “specifically binds” or “specifically binding” or “binds” refer to an antibody binding to an antigen or an epitope within the antigen with greater affinity than for other antigens. Typically, the antibody binds to the antigen or the epitope within the antigen with an equilibrium dissociation constant (K_{D}) of about 5×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein). The dissociation constant may be measured using known protocols. Antibodies that bind to the antigen or the epitope within the antigen may, however, have cross-reactivity to other related antigens, for example to the same antigen from other species (homologs), such as human or monkey, for example Macaca fascicularis (cynomolgus, cyno) or Pan troglodytes (chimpanzee, chimp). While a monospecific antibody binds one antigen or one epitope, a bispecific antibody binds two distinct antigens or two distinct epitopes.

“Complementarity determining regions” (CDR) are antibody regions that bind an antigen. CDRs may be defined using various delineations such as Kabat (Wu et al. (1970) J Exp Med 132:211-50) (Kabat et al., Sequences of Proteins of Immunological Interest, 5th Ed. Public Health Service, National Institutes of Health, Bethesda, Md., 1991), Chothia (Chothia et al. (1987) J Mol Biol 196:901-17), IMGT (Lefranc et al. (2003) Dev Comp Immunol 27:55-77) and AbM (Martin and Thornton (1996) J Bmol Biol 263:800-15). The correspondence between the various delineations and variable region numbering are described (see e.g., Lefranc et al. (2003) Dev Comp Immunol 27:55-77; Honegger and Pluckthun, (2001) J Mol Biol 309:657-70; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification.

“Full-length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Antigen binding fragment” refers to a portion of an immunoglobulin molecule that binds an antigen. Antigen binding fragments may be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

“Humanized antibodies” refers to antibodies in which the antigen binding sites are derived from non-human species and the variable region frameworks are derived from human immunoglobulin sequences. Humanized antibodies may include intentionally introduced mutations in the framework regions so that the framework may not be an exact copy of expressed human immunoglobulin or germline gene sequences.

“Human antibodies” refers to antibodies having heavy and light chain variable regions in which both the framework and the antigen binding site are derived from sequences of human origin. If the antibody contains a constant region or a portion of the constant region, the constant region is also derived from sequences of human origin. Antibodies in which antigen binding sites are derived from a non-human species are not included in the definition of “human antibody.”

A human antibody comprises heavy or light chain variable regions that are derived from sequences of human origin if the variable regions of the antibody are obtained from a system that uses human germline immunoglobulin or rearranged immunoglobulin genes. Non-limiting example systems include human immunoglobulin gene libraries displayed on phage, and transgenic non-human animals such as mice or rats carrying human immunoglobulin loci. A human antibody typically contains amino acid differences when compared to the human germline or rearranged immunoglobulin sequences due to, for example, naturally occurring somatic mutations, intentional substitutions in the framework or antigen binding site, and substitutions introduced during cloning or VDJ recombination in non-human animals. Typically, a human antibody is at least 80% identical in amino acid sequence to an amino acid sequence encoded by a human germline or rearranged immunoglobulin gene. For example, about: 80%, 81%, 82%, 83%, 84%, 85%, 86%, 87%, 88%, 89%, 90%, 91%, 92%, 93%, 94%, 95%, 96%, 97%, 98%, 99% or 100% identical. In some cases, a human antibody may contain consensus framework sequences derived from human framework sequence analyses (see, e.g., Knappik et al., J. Mol. Biol. 296:57-86 (2000)), or synthetic HCDR3 incorporated into human immune-globulin gene libraries displayed on phage (see, e.g., Shi et al., J. Mol. Biol. 397:385-96 (2010) and Int. Pat. Publ. No. WO2009/085462).

“Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Bispecific anti-EGFR/c-Met antibody” or “bispecific EGFR/c-Met antibody” refers to a bispecific antibody having a first domain that specifically binds EGFR and a second domain that specifically binds c-Met. The domains specifically binding EGFR and c-Met are typically VH/VL pairs, and the bispecific anti-EGFR/c-Met antibody is monovalent in terms of binding to EGFR and c-Met.

“Isolated” refers to a homogenous population of molecules (such as synthetic polynucleotides, polypeptides vectors or viruses) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Low fucose” or “low fucose content” as used in the application refers to antibodies with fucose content of about between 1%-15%. In some embodiments, the low fucose antibody comprises a biantennary glycan structure with a fucose content of less than about 20%.

“Normal fucose” or ‘normal fucose content” as used herein refers to antibodies with fucose content of about over 50%, typically about over 80% or over 85%.

“Recombinant” refers to DNA, antibodies and other proteins that are prepared, expressed, created or isolated by recombinant means when segments from different sources are joined to produce recombinant DNA, antibodies or proteins.

“Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used to formulate the bispecific anti-EGFR/c-Met antibody. These solutions are sterile and generally free of particulate matter. They may be sterilized by conventional, well-known sterilization techniques (e.g., filtration). For parenteral administration, the carrier may comprise sterile water and other excipients may be added to increase solubility or preservation. Injectable suspensions or solutions may also be prepared utilizing aqueous carriers along with appropriate additives. Suitable vehicles and formulations, inclusive of other human proteins, e.g., human serum albumin, are described, for example, in e.g., Remington: The Science and Practice of Pharmacy, 21st Edition, Troy, D. B. ed., Lipincott Williams and Wilkins, Philadelphia, PA 2006, Part 5, Pharmaceutical Manufacturing pp 691-1092, See especially pp. 958-989.

“Dosage” refers to the information of the amount of the therapeutic or the drug to be taken by the subject and the frequency of the number of times the therapeutic is to be taken by the subject. “Dose” refers to the amount or quantity of the therapeutic or the drug to be taken each time.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

“Co-administration,” “administration with,” “administration in combination with,” “in combination with” or the like, encompass administration of the selected therapeutics or drugs to a single patient, and are intended to include treatment regimens in which the therapeutics or drugs are administered by the same or different route of administration or at the same or different time.

“Fixed combination” refers to a single pharmaceutical composition comprising two or more compounds.

“Non-fixed combination” refers to separate pharmaceutical compositions, wherein each comprises one or more compounds. The one or more compounds or unit dosage forms can be administered as separate entities either simultaneously, concurrently or sequentially with no specific intervening time limits, wherein such administration provides effective levels of the two compounds in the body of the subject.

“Antagonist” or “inhibitor” refers to a molecule that, when bound to a cellular protein, suppresses at least one reaction or activity that is induced by a natural ligand of the protein. A molecule is an antagonist when the at least one reaction or activity is suppressed by at least about 20%, 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist.

“Treat”, “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Prevent”, “preventing”, “prevention”, or “prophylaxis” of a disease or disorder means preventing that a disorder occurs in subject.

“Responsive”, “responsiveness” or “likely to respond” refers to any kind of improvement or positive response, such as alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” are used interchangeably herein.

“Cancer” refers to an abnormal growth of cells which tend to proliferate in an uncontrolled way and, in some cases, to metastasize (spread) to other areas of a patient's body.

“EGFR or c-Met expressing cancer” refers to cancer that has detectable expression of EGFR or c-Met or has EGFR or c-Met mutation or amplification. EGFR or c-Met expression, amplification and mutation status can be detected using know methods, such as sequencing, next generation sequencing, fluorescent in situ hybridization, immunohistochemistry, flow cytometry or western blotting.

“Epidermal growth factor receptor” or “EGFR” refers to the human EGFR (also known as HER1 or ErbB1 (Ullrich et al., Nature 309:418-425, 1984) having the amino acid sequence shown in GenBank accession number NP_005219, as well as naturally-occurring variants thereof.

“Hepatocyte growth factor receptor” or “c-Met” or “MET” as used herein refers to the human c-Met having the amino acid sequence shown in GenBank Accession No: NP_001120972 and natural variants thereof.

“Newly diagnosed” refers to a subject who has been diagnosed with head and neck cancer, such as HNSCC, but has not yet received treatment for HNSCC.

“Treatment-naïve” refers to a subject who has not received treatment with a particular therapy. Treatment-naïve includes not receiving a systemic therapy in the recurrent and/or metastatic setting. Treatment-naïve includes “Treatment-naïve in the recurrent and/or metastatic setting” which refers to a subject who has not received a treatment with a particular therapy since receiving the diagnosis of the recurrent and/or metastatic cancer.

“Metastatic” refers to a cancer wherein cancer cells separate from the original tumor, enter the bloodstream or lymph system and spread to other areas of the body.

“Refractory” or “Resistant” refers to a disease that does not respond to a treatment. A refractory disease can be resistant to a treatment before or at the beginning of the treatment, or a refractory disease can become resistant during a treatment.

“Recurrent” or “Relapsed” refers to the return of a disease or the signs and symptoms of a disease after a period of improvement after prior treatment with a therapeutic.

“Diagnosing” or “diagnosis” refers to methods to determine if a subject is suffering from a given disease or condition or may develop a given disease or condition in the future or is likely to respond to treatment for a prior diagnosed disease or condition, i.e., stratifying a patient population on likelihood to respond to treatment. Diagnosis is typically performed by a physician based on the general guidelines for the disease to be diagnosed or other criteria that indicate a subject is likely to respond to a particular treatment.

“HPV unrelated” head and neck cancer refers to cancers that are not associated with human papillomavirus (HPV), high-risk HPV, negative for HPV, or otherwise do not test positive for HPV. For example, high-risk HPV types, such as for example HPV-16, 18, 31, 33, 35, 39, 45, 51, 52, 56, 58, or 59, are linked to a higher risk of developing oropharyngeal squamous cell carcinoma and other head and neck cancers. In contrast, cancers that do not involve HPV are considered to be HPV-independent or negative.

“Biological sample” refers to a collection of similar fluids, cells, or tissues isolated from a subject, as well as fluids, cells, or tissues present within a subject. Exemplary samples are biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, synovial fluid, liquid solutions contacted with a subject or biological source, for example, cell and organ culture medium including cell or organ conditioned medium, lavage fluids and the like, tissue biopsies, tumor tissue biopsies, tumor tissue samples, fine needle aspirations, surgically resected tissue, organ cultures or cell cultures.

“PD-L1 Combined Positive Score” or “CPS” refers to a scoring method evaluating the number of PD-L1-staining cells (tumor cells, lymphocytes, macrophages) relative to all viable tumor cells. CPS equals the [number of PD-L1 positive cells] divided by [total number of PD-L1 positive+PD-L1 negative tumor cells] multiplied by 100. Even though, the result of the CPS calculation can exceed 100, the maximum score is defined as CPS of 100. A minimum of 100 viable tumor cells in the PD-L1 stained slide is required for the specimen to be considered adequate for PD-L1 evaluation. In some embodiments, the number of PD-L1 staining cells is determined by immunohistochemistry.

“Hyaluronidase” refers to a class of enzymes that degrade hyaluronan. Hyaluronidases are endoglycosidases used to increase the dispersion and absorption of other co-administered drugs when administered subcutaneously (e.g., subcutaneous injections, subcutaneous infusion such as hypodermoclysis). Hyaluronidases include, but are not limited to, bacterial hyaluronidases (EC 4.2.2.1 or EC 4.2.99.1), hyaluronidases from leeches, other parasites and crustaceans (EC 3.2.1.36), and mammalian-type hyaluronidases (EC 3.2.1.35). Hyaluronidase (recombinant human) has a molecular weight of approximately 61 kDa. Hyaluronidases include those of non-human origin including, but not limited to, murine, canine, feline, leporine, avian, bovine, ovine, porcine, equine, piscine, ranine, bacterial, and any from leeches, other parasites, and crustaceans. Exemplary human hyaluronidases include HYALI, HYAL2, HYAL3, HYAL4, and PH20. Also included amongst hyaluronidases are soluble hyaluronidases, including, ovine and bovine PH20, and soluble forms of PH20. Exemplary hyaluronidases include those set forth in U.S. Pub. No. 2013/0302275, which is incorporated by reference herein, including, for example, hyaluronidases set forth as SEQ ID NOs: 6, 7-31, 69, 70, 71, 72, 856-861, 869-921 from U.S. Pub. No. 2013/0302275 (which are incorporated herein by reference), mature forms thereof (lacking the signal sequence), allelic variants thereof, and truncated forms thereof that exhibit hyaluronidase activity, including C-terminal truncated variants that are soluble.

As used herein, PH20 refers to a type of hyaluronidase that occurs in sperm and is neutral-active.

Soluble PH20 refers to a polypeptide characterized by its solubility under physiological conditions. Generally, a soluble PH20 lacks all or a portion of a glycophosphatidyl anchor (GPI) attachment sequence, or does not otherwise sufficiently anchor to the cell membrane. For example, a soluble PH20 can be a C-terminally truncated variant of a PH20 lacking a contiguous sequence of amino acids that corresponds to all or a portion of a GPI anchor attachment sequence. Hence, upon expression from a cell, a soluble PH20 is secreted into the medium.

Soluble human PH20 (sHuPH20) includes human PH20 polypeptides that lack a contiguous sequence of amino acids from the C-terminus of human PH20 that includes all or a portion of the GPI anchor sequence (C-terminally truncated PH20 polypeptides) such that upon expression, the polypeptides are soluble under physiological conditions. For example, soluble human PH20 polypeptides are C-terminally truncated polypeptides of human PH20 in its precursor form or in its mature form lacking the signal sequence, or allelic variants thereof as set forth in SEQ ID NOs: 36-42 herein and as disclosed in U.S. Pub. No. 20130302275 as SEQ ID NOs: 6, 7, and 68-72 of that reference which are incorporated herein by reference.

U.S. Pub. No. 2004/0268425, which is incorporated by reference herein, describes members of the soluble, neutral active Hyaluronidase Glycoprotein family, particularly the human soluble PH-20 Hyaluronidase Glycoproteins (also referred to as sHASEGPs). U.S. Pub. No. 20100143457, which is incorporated herein by reference, describes shorter active soluble PH20 (i.e., 36-469, -470, 471, and longer forms i.e., 36-495 to 36-500) the sequences of which are also incorporated herein by reference.

Hyaluronidase activity refers to the ability to enzymatically catalyze the cleavage of hyaluronic acid. The United States Pharmacopeia (USP) XXII assay for hyaluronidase determines hyaluronidase activity indirectly by measuring the amount of higher molecular weight hyaluronic acid, or hyaluronan, (HA) substrate remaining after the enzyme is allowed to react with the HA for 30 min at 37° C. (USP XXII-NF XVII (1990) 644-645 United States Pharmacopeia Convention, Inc, Rockville, MD). A Reference Standard solution can be used in an assay to ascertain the relative activity, in units, of any hyaluronidase. In vitro assays to determine the hyaluronidase activity of hyaluronidases, such as PH20, including modified PH20 polypeptides, are known in the art and described herein. Exemplary assays include the microturbidity assay that measures cleavage of hyaluronic acid by hyaluronidase indirectly by detecting the insoluble precipitate formed when the uncleaved hyaluronic acid binds with serum albumin. Reference Standards can be used, for example, to generate a standard curve to determine the activity in Units of the hyaluronidase being tested.

Specific activity refers to Units of activity per mg protein. The milligrams of hyaluronidase is defined by the absorption of a solution of at 280 nm assuming a molar extinction coefficient of approximately 1.7, in units of M-1 cm-1.

Neutral active refers to the ability of a PH20 polypeptide to enzymatically catalyze the cleavage of hyaluronic acid at neutral pH, such as at a pH between or about between pH 6.0 to pH 7.8.

Human recombinant DNA-derived hyaluronidase enzyme PH20 (rHuPH20) is a glycosylated single-chain protein produced by CHO cells containing a DNA plasmid encoding residues 36-482 of SEQ ID NO: 36, resulting in a heterogeneous mixture of soluble forms of human hyaluronidase (PH20) that begin at residue 36 and terminate at residues 478, 479, 480, 481, and 482 of SEQ ID NO: 36, including any one of SEQ ID NOs: 31, 35, 34, 33 and 32.

“Variant” refers to a polypeptide or a polynucleotide that differs from a reference polypeptide or a reference polynucleotide by one or more modifications for example, substitutions, insertions, or deletions.

### Methods of the Disclosure

It is well-known in the art that drug development is an unpredictable field. The lack of predictability in the art is evidenced, for example, by health authority requirements (such as those of the Food and Drug Administration) to establish a safe and effective dosing regimen for each individual drug candidate in clinical trials. Over the last decade (2011-2020), only 7.9% of all developmental drug candidates achieved FDA approval from a Phase I clinical study. See Clinical Development Success Rates and Contributing Factors 2011-2020. The rate of success is even lower in oncology, such that only 5.3% of oncology drug candidates succeed.

In the field of oncology, even for a drug that already has an established dose in a particular indication, the Food and Drug Administration (FDA) recommends further clinical studies to identify an optimal dose for a new indication; otherwise, patients may be exposed to unreasonable and significant risk, among other potential deficiencies. See, e.g., Optimizing the Dosage of Human Prescription Drugs and Biological Products for the Treatment of Oncologic Diseases; Draft Guidance for Industry; January 2023).

Despite recent advances in the treatment of HNSCC, patients continue to experience significant morbidity and mortality, underscoring the need to improve therapeutic approaches. EGFR- and c-Met-activating mutations are infrequently observed in HNSCC. However, EGFR and c-Met (MET) overexpression are common and are observed in approximately 90% and 80% of HNSCC tumors, respectively. While cetuximab-based therapies (anti-EGFR therapies) are utilized within HNSCC, providing an approximate ORR of 10% in the post-platinum population (Vermorken 2007; Cohen 2006), crosstalk between EGFR and c-Met signaling is known to promote resistance to anti-EGFR therapies in multiple cancers, including HNSCC. In addition, HGF/c-Met signaling promotes HNSCC growth and metastasis. However, targeting both EGFR and c-Met is not currently used in HNSCC treatment.

In one aspect, the disclosure provides a method of treating HNSCC (e.g., recurrent or metastatic head and neck squamous cell carcinoma) in a subject in need thereof, comprising administering a therapeutically effective amount of an anti-epidermal growth factor receptor (EGFR)/hepatocyte growth factor receptor (c-Met) antibody to the subject.

### Anti-EGFR/c-Met Antibodies

In some embodiments, the anti-EGFR/c-Met antibody is a bispecific antibody. In certain embodiments, the antibody is an isolated antibody. In particular embodiments, the antibody is an isolated bispecific antibody.

In some embodiments, the antibody (e.g., bispecific antibody) comprises a first domain that specifically binds EGFR and a second domain that specifically binds c-Met.

### EGFR Binding Arm

In some embodiments, the first domain that specifically binds EGFR comprises:
a) heavy chain complementarity determining region 1 (HCDR1), HCDR2, HCDR3 amino acid sequences of SEQ ID NOs: 1, 2 and 3, respectively; and/or
b) light chain complementarity determining region 1 (LCDR1), LCDR2 and LCDR3 amino acid sequences of SEQ ID NOs: 4, 5 and 6, respectively.

In certain embodiments, the first domain that specifically binds EGFR comprises:
a) HCDR1, HCDR2, HCDR3 amino acid sequences of SEQ ID NOs: 1, 2 and 3, respectively; and
b) LCDR1, LCDR2 and LCDR3 amino acid sequences of SEQ ID NOs: 4, 5 and 6, respectively.

HCDR1:
(SEQ ID NO: 1)
TYGMH
HCDR2:
(SEQ ID NO: 2)
VIWDDGSYKYYGDSVKG
HCDR3:
(SEQ ID NO: 3)
DGITMVRGVMKDYFDY
HCDR1:
(SEQ ID NO: 4)
RASQDISSALV
HCDR2:
(SEQ ID NO: 5)
DASSLES
HCDR3:
(SEQ ID NO: 6)
QQFNSYPLT

In some embodiments, the first domain comprises a heavy chain variable region (VH) amino acid sequence that is at least 90% identical to SEQ ID NO:13, e.g., about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:13. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%. In particular embodiments, the first domain comprises a VH of SEQ ID NO:13.

In certain embodiments, the first domain comprises a light chain variable region (VL) amino acid sequence that is at least 90% identical to SEQ ID NO:14, e.g., about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:14. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%. In particular embodiments, the first domain comprises a VL of SEQ ID NO:14.

As used herein, the term “identical” or “has sequence identity,” refers to the extent to which two amino acid sequences have the same residues at the same positions when the sequences are aligned to achieve a maximal level of identity, expressed as a percentage. For sequence alignment and comparison, typically one sequence is designated as a reference sequence, to which a test sequences are compared. The sequence identity between reference and test sequences is expressed as the percentage of positions across the entire length of the reference sequence where the reference and test sequences share the same amino acid upon alignment of the reference and test sequences to achieve a maximal level of identity. As an example, two sequences are considered to have 70% sequence identity when, upon alignment to achieve a maximal level of identity, the test sequence has the same amino acid residue at 70% of the same positions over the entire length of the reference sequence.

In some embodiments, the first domain comprises:
a) a VH amino acid sequence that is at least 90% identical to SEQ ID NO:13; and/or
b) a VL amino acid sequence that is at least 90% identical to SEQ ID NO:14.

In some embodiments, the first domain comprises:
a) a VH amino acid sequence that is at least 90% identical to SEQ ID NO:13; and
b) a VL amino acid sequence that is at least 90% identical to SEQ ID NO:14.

In some embodiments, the first domain comprises:
a) a VH of SEQ ID NO: 13; and/or
b) a VL of SEQ ID NO:14.

In particular embodiments, the first domain comprises:
a) a VH of SEQ ID NO:13; and
b) a VL of SEQ ID NO:14.

VH:
(SEQ ID NO: 13)
QVQLVESGGGVVQPGRSLRLSCAASGFTFSTYGMHWVRQAPGKGLEW
VAVIWDDGSYKYYGDSVKGRFTISRDNSKNTLYLQMNSLRAEDTAVYYC
ARDGITMVRGVMKDYFDYWGQGTLVTVSS
VL:
(SEQ ID NO: 14)
AIQLTQSPSSLSASVGDRVTITCRASQDISSALVWYQQKPGKAPKLLIY
DASSLESGVPSRFSGSESGTDFTLTISSLQPEDFATYYCQQFNSYPLTF
GGGTKVEIK

In some embodiments, the first domain comprises a first heavy chain (HCl) amino acid sequence that is at least 80% identical to SEQ ID NO:17, e.g., about: 81%, 82%, 83%, 84%, 85%, 86%, 87%, 88%, 89%, 90%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO: 17. In certain embodiments, the sequence identity is about: 80-99.9%, 80-99.8%, 85-99.8%, 85-99.6%, 90-99.6%, 90-99.5%, 95-99.5%, 95-99.4%, 96-99.4%, 96-99.2%, 97-99.2% or 97-99%. In particular embodiments, the first domain comprises a HCl amino acid sequence of SEQ ID NO: 17.

In some embodiments, the first domain comprises a first light chain (LC1) amino acid sequence that is at least 80% identical to SEQ ID NO:18, e.g., about: 81%, 82%, 83%, 84%, 85%, 86%, 87%, 88%, 89%, 90%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO: 18. In certain embodiments, the sequence identity is about: 80-99.9%, 80-99.8%, 85-99.8%, 85-99.6%, 90-99.6%, 90-99.5%, 95-99.5%, 95-99.4%, 96-99.4%, 96-99.2%, 97-99.2% or 97-99%. In particular embodiments, the first domain comprises a LC1 amino acid sequence of SEQ ID NO: 18.

In some embodiments, the first domain comprises:
a) a HCl amino acid sequence that is at least 80% identical to SEQ ID NO:17; and/or
b) a LC1 amino acid sequence that is at least 80% identical to SEQ ID NO:18.

In some embodiments, the first domain comprises:
a) a HCl amino acid sequence that is at least 80% identical to SEQ ID NO:17; and
b) a LC1 amino acid sequence that is at least 80% identical to SEQ ID NO:18.

In some embodiments, the first domain comprises:
a) a HCl of SEQ ID NO:17; and/or
b) a LC1 of SEQ ID NO:18.

In some embodiments, the first domain comprises:
a) a HCl of SEQ ID NO:17; and
b) a LC1 of SEQ ID NO:18.

HC1:
(SEQ ID NO: 17)
QVQLVESGGGVVQPGRSLRLSCAASGFTFSTYGMHWVRQAPGKGLE
WVAVIWDDGSYKYYGDSVKGRFTISRDNSKNTLYLQMNSLRAEDTAVYY
CARDGITMVRGVMKDYFDYWGQGTLVTVSSASTKGPSVFPLAPSSKSTS
GGTAALGCLVKDYFPEPVTVSWNSGALTSGVHTFPAVLQSSGLYSLSSV
VTVPSSSLGTQTYICNVNHKPSNTKVDKRVEPKSCDKTHTCPPCPAPEL
LGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEW
ESNGQPENNYKTTPPVLDSDGSFLLYSKLTVDKSRWQQGNVFSCSVMHE
ALHNHYTQKSLSLSPGK
LC1:
(SEQ ID NO: 18)
AIQLTQSPSSLSASVGDRVTITCRASQDISSALVWYQQKPGKAPKLLIY
DASSLESGVPSRFSGSESGTDFTLTISSLQPEDFATYYCQQFNSYPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

c-Met (MET) Binding Arm

In certain embodiments, the second domain that specifically binds c-Met comprises:
a) HCDR1, HCDR2, HCDR3 amino acid sequences of SEQ ID NOs: 7, 8 and 9, respectively; and/or
b) LCDR1, LCDR2 and LCDR3 amino acid sequences of SEQ ID NOs: 10, 11 and 12, respectively.

In certain embodiments, the second domain that specifically binds c-Met comprises:
a) HCDR1, HCDR2, HCDR3 amino acid sequences of SEQ ID NOs: 7, 8 and 9, respectively; and
b) LCDR1, LCDR2 and LCDR3 amino acid sequences of SEQ ID NOs: 10, 11 and 12, respectively.

HCDR1:
(SEQ ID NO: 7)
SYGIS
HCDR2:
(SEQ ID NO: 8)
WISAYNGYTNYAQKLQG
HCDR3:
(SEQ ID NO: 9)
DLRGTNYFDY
HCDR1:
(SEQ ID NO: 10)
RASQGISNWLA
HCDR2:
(SEQ ID NO: 11)
AASSLLS
HCDR3:
(SEQ ID NO: 12)
QQANSFPIT

In some embodiments, the second domain comprises a VH amino acid sequence that is at least 90% identical to SEQ ID NO:15, e.g., about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:15. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%. In particular embodiments, the second domain comprises a VH of SEQ ID NO:15

In certain embodiments, the second domain comprises a VL amino acid sequence that is at least 90% identical to SEQ ID NO:16, e.g., about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:16. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%. In particular embodiments, the second domain comprises a VL of SEQ ID NO: 16.

In some embodiments, the second domain comprises:
a) a VH amino acid sequence that is at least 90% identical to SEQ ID NO:15; and/or
b) a VL amino acid sequence that is at least 90% identical to SEQ ID NO:16.

In some embodiments, the second domain comprises:
a) a VH amino acid sequence that is at least 90% identical to SEQ ID NO:15; and
b) a VL amino acid sequence that is at least 90% identical to SEQ ID NO:16.

In some embodiments, the second domain comprises:
a) a VH of SEQ ID NO: 15; and/or
b) a VL of SEQ ID NO:16.

In particular embodiments, the second domain comprises:
a) a VH of SEQ ID NO: 15; and
b) a VL of SEQ ID NO:16.

VH:
(SEQ ID NO: 15)
QVQLVQSGAEVKKPGASVKVSCETSGYTFTSYGISWVRQAPGHGLEW
MGWISAYNGYTNYAQKLQGRVTMTTDTSTSTAYMELRSLRSDDTAVYYC
ARDLRGTNYFDYWGQGTLVTVSS
VL:
(SEQ ID NO: 16)
DIQMTQSPSSVSASVGDRVTITCRASQGISNWLAWFQHKPGKAPKLLIY
AASSLLSGVPSRFSGSGSGTDFTLTISSLQPEDFATYYCQQANSFPITF
GQGTRLEIK

In some embodiments, the second domain comprises a second heavy chain (HC2) amino acid sequence that is at least 80% identical to SEQ ID NO:19, e.g., about: 81%, 82%, 83%, 84%, 85%, 86%, 87%, 88%, 89%, 90%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:19. In certain embodiments, the sequence identity is about: 80-99.9%, 80-99.8%, 85-99.8%, 85-99.6%, 90-99.6%, 90-99.5%, 95-99.5%, 95-99.4%, 96-99.4%, 96-99.2%, 97-99.2% or 97-99%. In particular embodiments, the second domain comprises a HC2 amino acid sequence of SEQ ID NO:19.

In some embodiments, the second domain comprises a second light chain (LC2) amino acid sequence that is at least 80% identical to SEQ ID NO:20, e.g., about: 81%, 82%, 83%, 84%, 85%, 86%, 87%, 88%, 89%, 90%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:20. In certain embodiments, the sequence identity is about: 80-99.9%, 80-99.8%, 85-99.8%, 85-99.6%, 90-99.6%, 90-99.5%, 95-99.5%, 95-99.4%, 96-99.4%, 96-99.2%, 97-99.2% or 97-99%. In particular embodiments, the second domain comprises a LC2 amino acid sequence of SEQ ID NO:20.

In some embodiments, the second domain comprises:
a) a HC2 amino acid sequence that is at least 80% identical to SEQ ID NO:19; and/or
b) a LC2 amino acid sequence that is at least 80% identical to SEQ ID NO:20.

In some embodiments, the second domain comprises:
a) a HC2 amino acid sequence that is at least 80% identical to SEQ ID NO:19; and
b) a LC2 amino acid sequence that is at least 80% identical to SEQ ID NO:20.

In some embodiments, the second domain comprises:
a) a HC2 of SEQ ID NO: 19; and/or
b) a LC2 of SEQ ID NO:20.

In some embodiments, the second domain comprises:
a) a HC2 of SEQ ID NO:19; and
b) a LC2 of SEQ ID NO:20.

HC2:
(SEQ ID NO: 19)
QVQLVQSGAEVKKPGASVKVSCETSGYTFTSYGISWVRQAPGHGLEW
MGWISAYNGYTNYAQKLQGRVTMTTDTSTSTAYMELRSLRSDDTAVYYC
ARDLRGTNYFDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKRVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPE
NNYKTTPPVLDSDGSFFLYSRLTVDKSRWQQGNVFSCSVMHEALHNHYT
QKSLSLSPGK
LC2:
(SEQ ID NO: 20)
DIQMTQSPSSVSASVGDRVTITCRASQGISNWLAWFQHKPGKAPKLLIY
AASSLLSGVPSRFSGSGSGTDFTLTISSLQPEDFATYYCQQANSFPITF
GQGTRLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

In some embodiments, the antibody (e.g., bispecific antibody) comprises:
a) a first domain that specifically binds EGFR, comprising HCDR1, HCDR2, HCDR3, LCDR1, LCDR2 and LCDR3 amino acid sequences of SEQ ID NOs: 1, 2, 3, 4, 5 and 6, respectively; and/or
b) a second domain that specifically binds c-Met, comprising HCDR1, HCDR2, HCDR3, LCDR1, LCDR2 and LCDR3 amino acid sequences of SEQ ID NOs: 7, 8, 9, 10, 11 and 12, respectively.

In certain embodiments, the antibody (e.g., bispecific antibody) comprises:
a) a first domain that specifically binds EGFR, comprising HCDR1, HCDR2, HCDR3, LCDR1, LCDR2 and LCDR3 amino acid sequences of SEQ ID NOs: 1, 2, 3, 4, 5 and 6, respectively; and
b) a second domain that specifically binds c-Met, comprising HCDR1, HCDR2, HCDR3, LCDR1, LCDR2 and LCDR3 amino acid sequences of SEQ ID NOs: 7, 8, 9, 10, 11 and 12, respectively.

In some embodiments, the antibody (e.g., bispecific antibody) comprises:
a) a first domain comprising a VH amino acid sequence that is at least 90% identical to SEQ ID NO:13;
b) a first domain comprising a VL amino acid sequence that is at least 90% identical to SEQ ID NO:14;
c) a second domain comprising a VH amino acid sequence that is at least 90% identical to SEQ ID NO:15; and/or
d) a second domain comprising a VL amino acid sequence that is at least 90% identical to SEQ ID NO:16.

In certain embodiments, the antibody (e.g., bispecific antibody) comprises:
a) a first domain comprising a VH amino acid sequence that is at least 90% identical to SEQ ID NO:13;
b) a first domain comprising a VL amino acid sequence that is at least 90% identical to SEQ ID NO:14;
c) a second domain comprising a VH amino acid sequence that is at least 90% identical to SEQ ID NO:15; and
d) a second domain comprising a VL amino acid sequence that is at least 90% identical to SEQ ID NO:16.

In some embodiments, the antibody (e.g., bispecific antibody) comprises:
a) a first domain comprising a VH of SEQ ID NO:13;
b) a first domain comprising a VL of SEQ ID NO:14;
c) a second domain comprising a VH of SEQ ID NO: 15; and/or
d) a second domain comprising a VL of SEQ ID NO:16.

In some embodiments, the antibody (e.g., bispecific antibody) comprises:
a) a first domain comprising a VH of SEQ ID NO:13;
b) a first domain comprising a VL of SEQ ID NO:14;
c) a second domain comprising a VH of SEQ ID NO: 15; and
d) a second domain comprising a VL of SEQ ID NO:16.

In certain embodiments, the antibody (e.g., bispecific antibody) comprises:
a) a HCl amino acid sequence that is at least 80% identical to SEQ ID NO:17;
b) a LC1 amino acid sequence that is at least 80% identical to SEQ ID NO:18;
c) a HC2 amino acid sequence that is at least 80% identical to SEQ ID NO:19; and/or
d) a LC2 amino acid sequence that is at least 80% identical to SEQ ID NO:20.

In certain embodiments, the antibody (e.g., bispecific antibody) comprises:
a) a HCl amino acid sequence that is at least 80% identical to SEQ ID NO:17;
b) a LC1 amino acid sequence that is at least 80% identical to SEQ ID NO:18;
c) a HC2 amino acid sequence that is at least 80% identical to SEQ ID NO:19; and
d) a LC2 amino acid sequence that is at least 80% identical to SEQ ID NO:20.

In certain embodiments, the antibody (e.g., bispecific antibody) comprises:
a) a HCl of SEQ ID NO:17;
b) a LC1 of SEQ ID NO:18;
c) a HC2 of SEQ ID NO: 19; and/or
d) a LC2 of SEQ ID NO:20.

In particular embodiments, the antibody (e.g., bispecific antibody) comprises:
a) a HCl of SEQ ID NO:17;
b) a LC1 of SEQ ID NO:18;
c) a HC2 of SEQ ID NO:19; and
d) a LC2 of SEQ ID NO:20.

In some embodiments, the antibody (e.g., bispecific antibody) is of the IgG isotype. In certain embodiments, the antibody (e.g., bispecific antibody) is of the IgG1 isotype. Some variation exists within the IgG1 constant domain (e.g., well-known allotypes), for example, with variation at positions 214, 356, 358, 422, 431, 435 and/or 436 (residue numbering according to the EU numbering) (see e.g., IMGT Web resources; IMGT Repertoire (IG and TR); Proteins and alleles; allotypes). The bispecific anti-EGFR/c-Met antibody may be of any IgG1 allotype, such as G1m17, G1m3, Glml, G1m2, G1m27 or G1m28.

In some embodiments, the antibody is a human antibody.

In particular embodiments, the antibody is amivantamab. Amivantamab or JNJ-61186372 (JNJ-372) is an IgG1 anti-EGFR/c-Met bispecific antibody described in U.S. Pat. No. 9,593,164. The disclosure is based, at least in part, on the finding that amivantamab has demonstrated robust anti-tumor activity in preclinical p16-negative patient-derived xenograft models of HNSCC, grown in mice.

Other anti-EGFR/c-Met antibodies (e.g., bispecific antibodies) may also be used in the methods of the disclosure, for example, by combining publicly available EGFR binding VH/VL domains and c-Met binding VH/VL domains.

In some embodiments, the antibody (e.g., bispecific antibody) comprises a biantennary glycan structure with a fucose content of between about 1% to about 15%. In some embodiments, the antibody comprises a biantennary glycan structure with a fucose content of less than about 20%.

Antibodies with reduced fucose content can be made using different methods reported to lead to the successful expression of relatively high defucosylated antibodies bearing the biantennary complex-type of Fc oligosaccharides such as control of culture osmolality (Konno et al., Cytotechnology 64 (: 249-65, 2012), application of a variant CHO line Lec13 as the host cell line (Shields et al., J Biol Chem 277:26733-26740, 2002), application of a variant CHO line EB66 as the host cell line (Olivier et al., MAbs; 2 (4), 2010; Epub ahead of print; PMID: 20562582), application of a rat hybridoma cell line YB2/0 as the host cell line (Shinkawa et al., J Biol Chem 278:3466-3473, 2003), introduction of small interfering RNA specifically against the α 1,6-fucosyltrasferase (FUT8) gene (Mori et al., Biotechnol Bioeng 88: 901-908, 2004), or coexpression of β-1,4-N-acetylglucosaminyltransferase III and Golgi α-mannosidase II or a potent alpha-mannosidase I inhibitor, kifunensine (Ferrara et al., J Biol Chem 281: 5032-5036, 2006, Ferrara et al., Biotechnol Bioeng 93:851-861, 2006; Zhou et al., Biotechnol Bioeng 99:652-65, 2008). In general, lowering fucose content in the glycan of the antibodies potentiates antibody-mediated cellular cytotoxicity (ADCC).

The composition can comprise an amount of hyaluronidase enzyme that results in an increase in the dispersion of the bispecific antibody during the subcutaneous administration. The hyaluronidase enzyme has no adverse effect on the molecular integrity of the bispecific antibody. Furthermore, the hyaluronidase enzyme merely modifies the delivery of the bispecific antibody to the systemic circulation but does not possess any properties that could provide or contribute to the therapeutic effects of systemically absorbed bispecific antibody. The hyaluronidase enzyme is not systemically bioavailable and does not adversely affect the molecular integrity of the bispecific antibody at the recommended storage conditions of the composition. A number of suitable hyaluronidase enzymes are known. The preferred enzyme is a human hyaluronidase enzyme, such as a soluble human PH20 hyaluronidase, preferably the recombinant human hyaluronidase enzyme product known as rHuPH20. The amino acid sequence of soluble human PH20 hyaluronidases include the soluble human PH20 known as rHuPH20 and available under CAS Registry No. 757971-58-7. Soluble human PH20 hyaluronidases are described in Int'l Pub. No. WO2004/078140 and U.S. Pat. No. 7,767,429 incorporated herein by reference, in their entirety. In some embodiments, soluble hyaluronidases include those whose sequence are set forth in any of SEQ ID NOs: 31-35. Soluble PH20 hyaluronidase, when expressed in a cell, include a signal sequence for trafficking in the cell. Thus, in some embodiments, the amino acid sequence of the soluble PH20 hyaluronidase comprises SEQ ID NO: 36. In some embodiments, the amino acid sequence of the soluble PH20 hyaluronidase comprises SEQ ID NO: 32, namely residues 36-482 of wild type human hyaluronidase. In some embodiments, the amino acid sequence of the soluble PH20 hyaluronidase comprises SEQ ID NO: 33. In some embodiments, the amino acid sequence of the soluble PH20 hyaluronidase comprises SEQ ID NO: 34. In some embodiments, the soluble PH20 hyaluronidase comprises rHuPH20 comprising the amino acid sequence of SEQ ID NO: 35. In some embodiments, the amino acid sequence of the soluble PH20 hyaluronidase comprises SEQ ID NO: 31. In some embodiments, the soluble PH20 hyaluronidases, when expressed in a cell, comprise a mixture of species that can include any one of SEQ ID NOs: 31, 32, 33, 34, and 35 in various abundance. The average molecular weight is 61 kDa.

rHuPH20 refers to the composition produced upon expression in a cell, such as CHO cell, of nucleic acids that encode residues 36-482 of SEQ ID NO: 36, generally linked to the native or a heterologous signal sequence (residues 1-35 of SEQ ID NO: 36). rHuPH20 is produced by expression of a nucleic acid molecule, such as encoding amino acids 1-482 (set forth in SEQ ID NO: 36) in a mammalian cell. Translational processing removes the 35 amino acid signal sequence. As produced in the culture medium there is heterogeneity at the C-terminus such that the product, designated rHuPH20, includes a mixture of species that can include one or more of the polypeptides 36-480, 36-481, and 36-482 of SEQ ID NO: 36, and some shorter polypeptides, in various abundance. Typically, rHuPH20 is produced in cells, such as CHO cells, for example DG44 CHO cells, that facilitate correct N-glycosylation to retain activity. In some embodiments, the hyaluronidase is a soluble human PH20 (rHuPH20) comprising the amino acid sequence of SEQ ID NO: 35. In some embodiments, the hyaluronidase is a soluble human PH20 (rHuPH20) comprising the amino acid sequence of SEQ ID NO: 31. In some embodiments, the hyaluronidase is a soluble human PH20 (rHuPH20) comprising the amino acid sequence of SEQ ID NO: 32. In some embodiments, the hyaluronidase is a soluble human PH20 (rHuPH20) comprising the amino acid sequence of SEQ ID NO: 33. In some embodiments, the hyaluronidase is a soluble human PH20 (rHuPH20) comprising the amino acid sequence of SEQ ID NO: 34.

| | |
|---|---|
| SEQ ID NO: 31: | LNFRAPPVIPNVPFLWAWNAPSEFCLGKFDEPLDMSLFSFIGS |
| rHuPH20 | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDFLVETIKLG |
| | KLLRPNHLWGYYLFPDCYNHHYKKPGYNGSCENVEIKRNDDLS |
| | WLWNESTALYPSIYLNTQQSPVAATLYVRNRVREAIRVSKIPD |
| | AKSPLPVFAYTRIVFTDQVLKFLSQDELVYTFGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEP |
| SEQ ID NO: 32: | LNFRAPPVIPNVPFLWAWNAPSEFCLGKFDEPLDMSLFSFIGS |
| rHuPH20 variant 1 | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDFLVETIKLG |
| | KLLRPNHLWGYYLFPDCYNHHYKKPGYNGSCENVEIKRNDDLS |
| | WLWNESTALYPSIYLNTQQSPVAATLYVRNRVREAIRVSKIPD |
| | AKSPLPVFAYTRIVFTDQVLKELSQDELVYTFGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEPQIFY |
| SEQ ID NO: 33: | LNFRAPPVIPNVPFLWAWNAPSEFCLGKFDEPLDMSLFSFIGS |
| rHuPH20 variant 2 | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDFLVETIKLG |
| | KLLRPNHLWGYYLFPDCYNHHYKKPGYNGSCFNVEIKRNDDLS |
| | WLWNESTALYPSIYLNTQQSPVAATLYVRNRVREAIRVSKIPD |
| | AKSPLPVFAYTRIVFTDQVLKFLSQDELVYTFGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEPQIF |
| SEQ ID NO: 34: | LNFRAPPVIPNVPFLWAWNAPSEFCLGKFDEPLDMSLFSFIGS |
| rHuPH20 variant 3 | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDFLVETIKLG |
| | KLLRPNHLWGYYLFPDCYNHHYKKPGYNGSCENVEIKRNDDLS |
| | WLWNESTALYPSIYLNTQQSPVAATLYVRNRVREAIRVSKIPD |
| | AKSPLPVFAYTRIVFTDQVLKFLSQDELVYTFGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEPQI |
| SEQ ID NO: 35: | LNFRAPPVIPNVPFLWAWNAPSEFCLGKFDEPLDMSLFSFIGS |
| rHuPH20 variant 4 | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDFLVETIKLG |
| | KLLRPNHLWGYYLFPDCYNHHYKKPGYNGSCENVEIKRNDDLS |
| | WLWNESTALYPSIYLNTQQSPVAATLYVRNRVREAIRVSKIPD |
| | AKSPLPVFAYTRIVFTDQVLKFLSQDELVYTFGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEPQ |
| SEQ ID NO: 36 | MGVLKFKHIFFRSFVKSSGVSQIVFTFLLIPCCLTLNFRAPPV |
| | IPNVPFLWAWNAPSEFCLGKFDEPLDMSLFSFIGSPRINATGQ |
| | GVTIFYVDRLGYYPYIDSITGVTVNGGIPQKISLQDHLDKAKK |
| | DITFYMPVDNLGMAVIDWEEWRPTWARNWKPKDVYKNRSIELV |
| | QQQNVQLSLTEATEKAKQEFEKAGKDFLVETIKLGKLLRPNHL |
| | WGYYLFPDCYNHHYKKPGYNGSCFNVEIKRNDDLSWLWNESTA |
| | LYPSIYLNTQQSPVAATLYVRNRVREAIRVSKIPDAKSPLPVF |
| | AYTRIVFTDQVLKFLSQDELVYTFGETVALGASGIVIWGTLSI |
| | MRSMKSCLLLDNYMETILNPYIINVTLAAKMCSQVLCQEQGVC |
| | IRKNWNSSDYLHLNPDNFAIQLEKGGKFTVRGKPTLEDLEQFS |
| | EKFYCSCYSTLSCKEKADVKDTDAVDVCIADGVCIDAFLKPPM |
| | ETEEPQIFYNASPSTLSATMFIVSILFLIISSVASL |
| SEQ ID NO: 37 | LNFRAPPVIPNVPFLWAWNAPSEFCLGKFDEPLDMSLFSFIGS |
| | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDELVETIKLG |
| | KLLRPNHLWGYYLFPDCYNHHYKKPGYNGSCFNVEIKRNDDLS |
| | WLWNESTALYPSIYLNTQQSPVAATLYVRNRVREAIRVSKIPD |
| | AKSPLPVFAYTRIVFTDQVLKFLSQDELVYTFGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEPQIFYNASPSTLSATMFIVSILFLIISSVAS |
| | L |
| SEQ ID NO: 38 | MGVLKFKHIFFRSFVKSSGVSQIVFTFLLIPCCLTLNFRAPPV |
| | IPNVAFLWAWNAPSEFCLGKFDEPLDMSLFSFIGSPRINATGQ |
| | GVTIFYVDRLGYYPYIDSITGVTVNGGIPQKISLQDHLDKAKK |
| | DITFYMPVDNLGMAVIDWEEWRPTWARNWKPKDVYKNRSIELV |
| | QQQNVQLSLTEATEKAKQEFEKAGKDFLVETIKLGKLLRPNHL |
| | WGYYLFPDCYNHHYKKPGYNGSCFNVEIKRNDDLSWLWNESTA |
| | LYPSIYLNTQQSPVAATLYVRNRVREAIRVSKIPDAKSPLPVF |
| | AYTRIVFTDQVLKFLSQDELVYTFGETVALGASGIVIWGTLSI |
| | MRSMKSCLLLDNYMETILNPYIINVTLAAKMCSQVLCQEQGVC |
| | IRKNWNSSDYLHLNPDNFAIQLEKGGKFTVRGKPTLEDLEQFS |
| | EKFYCSCYSTLSCKEKADVKDTDAVDVCIADGVCIDAFLKPPM |
| | ETEEPQIFYNASPSTLSATMFIVSIWFLIISSVASL |
| SEQ ID NO: 39 | LNFRAPPVIPNVAFLWAWNAPSEFCLGKFDEPLDMSLFSFIGS |
| | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDFLVETIKLG |
| | KLLRPNHLWGYYLFPDCYNHHYKKPGYNGSCENVEIKRNDDLS |
| | WLWNESTALYPSIYLNTQQSPVAATLYVRNRVREAIRVSKIPD |
| | AKSPLPVFAYTRIVFTDQVLKFLSQDELVYTFGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEPQIFYNASPSTLSATMFIVSIWFLIISSVAS |
| | L |
| SEQ ID NO: 40 | MGVLQFKHIFFRSFVKSSGVSQIVFTFLLIPCCLTLNFRAPPV |
| | IPNVPFLWAWNAPSEFCLGKFDEPLDMSLFSFIGSPRINATGQ |
| | GVTIFYVDRLGYYPYIDSITGVTVNGGIPQKISLQDHLDKAKK |
| | DITFYMPVDNLGMAVIDWEEWRPTWARNWKPKDVYKNRSIELV |
| | QQQNVQLSLTEATEKAKQEFEKAGKDFLVETIKLGKLLRPNHL |
| | WGYYLFPDCYNHHYKKPGYNGSCFNVEIKRNDDLSWLWNESTA |
| | LYPSIYLNTQQSPVAATLYVRNRVREAIRVSKIPDAKSPLPVF |
| | AYTRIVFTDQVLKFLSQDELVYTFGETVALGASGIVIWGTLSI |
| | MRSMKSCLLLDNYMETILNPYIINVTLAAKMCSQVLCQEQGVC |
| | IRKNWNSSDYLHLNPDNFAIQLEKGGKFTVRGKPTLEDLEQFS |
| | EKFYCSCYSTLSCKEKADVKDTDAVDVCIADGVCIDAFLKPPM |
| | ETEEPQIFYNASPSTLSATMFIVSILFLIISSVASL |
| SEQ ID NO: 41 | MGVLKFKHIFFRSFVKSSGVSQIVFTFLLIPCCLTLNFRAPPV |
| | IPNAPFLWAWNAPSEFCLGKFDEPLDMSLFSFIGSPRINATGQ |
| | GVTIFYVDRLGYYPYIDSITGVTVNGGIPQKISLQDHLDKAKK |
| | DITFYMPVDNLGMAVIDWEEWRPTWARNWKPKDVYKNRSIELV |
| | QQQNVQLSLTEATEKAKQEFEKAGKDELVETIKLGKLLRPNHL |
| | WGYYLFPDCYNHHYKKPGYNGSCFNVEIKRNDDLSWLWNESTA |
| | LYPSIYLNTQQSPVAATLYVRNRVREAIRVSKIPDAKSPLPVE |
| | AYTRIVFTDQVLKFLSQDELVYTFGETVALGASGIVIWGTLSI |
| | MRSMKSCLLLDNYMETILNPYIINVTLAAKMCSQVLCQEQGVC |
| | IRKNWNSSDYLHLNPDNFAIQLEKGGKFTVRGKPTLEDLEQFS |
| | EKFYCSCYSTLSCKEKADVKDTDAVDVCIADGVCIDAFLKPPM |
| | ETEEPQIFYNASPSTLSATMFIVSILFLIISSVASL |
| SEQ ID NO: 42 | LNFRAPPVIPNAPFLWAWNAPSEFCLGKFDEPLDMSLFSFIGS |
| | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDELVETIKLG |
| | KLLRPNHLWGYYLFPDCYNHHYKKPGYNGSCENVEIKRNDDLS |
| | WLWNESTALYPSIYLNTQQSPVAATLYVRNRVREAIRVSKIPD |
| | AKSPLPVFAYTRIVFTDQVLKFLSQDELVYTFGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEPQIFYNASPSTLSATMFIVSILFLIISSVAS |
| | L |

### Generating Anti-EGFR/c-Met Antibodies

Anti-EGFR/c-Met antibodies used in the methods of the disclosure may be generated, for example, using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms may be engineered to favor heterodimerization over homodimerization. The resulting product is a bispecific antibody having two Fab arms or half molecules which each bind a distinct epitope, i.e., an epitope on EGFR and an epitope on c-Met. For example, the bispecific antibodies of the invention may be generated using the technology described in Int. Pat. Publ. No. WO2011/131746. Mutations F405L in one heavy chain and K409R in the other heavy chain may be used in case of IgG1 antibodies. For IgG2 antibodies, a wild-type IgG2 and a IgG2 antibody with F405L and R409K substitutions may be used. For IgG4 antibodies, a wild-type IgG4 and a IgG4 antibody with F405L and R409K substitutions may be used. To generate bispecific antibodies, the first monospecific bivalent antibody and the second monospecific bivalent antibody are engineered to have the aforementioned mutation in the Fc region, and the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Bispecific anti-EGFR/c-Met antibodies used in the methods of the disclosure may also be generated using designs such as the Knob-in-Hole (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody) (EMD Serono), and the Biclonic (Merus).

In the “knob-in-hole” strategy (see, e.g., Intl. Publ. No. WO 2006/028936) select amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

CrossMAb technology, in addition to utilizing the “knob-in-hole” strategy to promote Fab arm exchange utilizes CHI/CL domain swaps in one half arm to ensure correct light chain pairing of the resulting bispecific antibody (see e.g., U.S. Pat. No. 8,242,247).

Other cross-over strategies may be used to generate full length bispecific antibodies of the invention by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in Int. Patent Publ. Nos. WO2009/080254, WO2009/080251, WO2009/018386 and WO2009/080252.

Other strategies such as promoting heavy chain heterodimerization using electrostatic interactions by substituting positively charged residues at one CH3 surface and negatively charged residues at a second CH3 surface may be used, as described in US Patent Publ. No. US2010/0015133; US Patent Publ. No. US2009/0182127; US Patent Publ. No. US2010/028637 or US Patent Publ. No. US2011/0123532. In other strategies, heterodimerization may be promoted by the following substitutions (expressed as modified positions in the first CH3 domain of the first heavy chain/modified position in the second CH3 domain of the second heavy chain): L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in U.S. Patent Publ. No. US2012/0149876 or U.S. Patent Publ. No. US2013/0195849.

SEEDbody technology may be utilized to generate bispecific antibodies of the invention. SEEDbodies have, in their constant domains, select IgG residues substituted with IgA residues to promote heterodimerization as described in U.S. Patent No. US20070287170.

Mutations are typically made at the DNA level to a molecule such as the constant domain of the antibody using standard methods.

### Administration

The anti-EGFR/c-Met antibody (e.g., bispecific antibody) and/or additional therapeutic (e.g., chemotherapeutic) agent may be administered in a pharmaceutical composition or compositions. In some embodiments, the pharmaceutical composition further comprises a pharmaceutically acceptable carrier.

The methods for subcutaneous formulation and administration of the anti-EGFR/c-Met antibody, such as amivantamab, are described in WO2022224187, which are incorporated herein by reference.

The mode of administration may be any suitable route that delivers the antibody (e.g., bispecific antibody) to the subject in need thereof, such as parenteral administration, e.g., intradermal, intramuscular, intraperitoneal, intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by, for example intratumoral, intracolic, intraabdominal, intragastric, intracavitary, intrapelvic, intraperitoneal, intrarectal, intrathoracic, intravascular, intralesional, rectal, buccal, sublingual, intranasal, or transdermal delivery.

In some embodiments, the pharmaceutical composition comprising the anti-EGFR/c-Met antibody (e.g., bispecific antibody) is administered via an intravenous infusion. In some embodiments, the additional therapeutic (e.g., chemotherapeutic) agent is administered via an intravenous infusion.

In some embodiments, the pharmaceutical composition comprising the anti-EGFR/c-Met antibody (e.g., bispecific antibody) is administered via a subcutaneous injection.

In some embodiments, the antibody (e.g., bispecific antibody) is administered at a dose of about 140 mg to about 3,360 mg, for example, about 700 mg to about 1,400 mg, about 700 mg to about 1,050 mg, about 1,050 mg to about 1,400 mg, about 1,750 mg to about 2,100 mg, about 1,600 mg to about 2240 mg, about 1,600 mg to about 3,360 mg, or about 2,240 mg to about 3,360 mg.

In some embodiments, the antibody (e.g., bispecific antibody) is administered at a dose of about: 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1,000, 1,010, 1,020, 1,030, 1,040, 1,050, 1,060, 1,070, 1,080, 1,090, 1,100, 1,110, 1,120, 1,130, 1,140, 1,150, 1,160, 1,170, 1,180, 1,190, 1,200, 1,210, 1,220, 1,230, 1,240, 1,250, 1,260, 1,270, 1,280, 1,290, 1,300, 1,310, 1,320, 1,330, 1,340, 1,350, 1,360, 1,370, 1,380, 1,390, 1,400, 1,410, 1,420, 1,430, 1,440, 1,450, 1,460, 1,470, 1,480, 1,490, 1,500, 1,510, 1,520, 1,530, 1,540, 1,550, 1,560, 1,570, 1,580, 1,590, 1,600, 1,610, 1,620, 1,630, 1,640, 1,650, 1,660, 1,670, 1,680, 1,690, 1,700, 1,710, 1,720, 1,730, 1,740, 1,750, 1,760, 1,770, 1,780, 1,790, 1,800, 1,810, 1,820, 1,830, 1,840, 1,850, 1,860, 1,870, 1,880, 1,890, 1,900, 1,910, 1,920, 1,930, 1,940, 1,950, 1,960, 1,970, 1,980, 1,990, 2,000, 2,010, 2,020, 2,030, 2,040, 2,050, 2,060, 2,070, 2,080, 2,090, 2,100, 2,110, 2,120, 2,130, 2,140, 2,150, 2,200, 2,230, 2,240, 2,250, 2,300, 2,350, 2,400, 2,450, 2,500, 2,550, 2,600, 2,650, 2,700, 2,750, 2,800, 2,850, 2,900, 3,000, 3,100, 3,200, 3,300, 3,350, 3,360, or 3,370 mg.

In some embodiments, the antibody is administered at a dose of about 700 mg, about 1,050 mg, about 1,400 mg, about 1,750 mg, about 2,100 mg, about 1,600 mg, about 2,240 mg, about 2,400 mg or about 3,360 mg. In some embodiments, the antibody is administered at a dose of about 1,050 mg. In certain embodiments, the antibody is administered at a dose of about 1,400 mg. In particular embodiments, the antibody is administered at a dose of about 700 mg. In some embodiments, the antibody is administered at a dose of about 1,750 mg. In some embodiments, the antibody is administered at a dose of about 1,600 mg. In some embodiments, the antibody is administered at a dose of about 2,100 mg. In some embodiments, the antibody is administered at a dose of about 2,240 mg. In some embodiments, the antibody is administered at a dose of about 2,400 mg. In some embodiments, the antibody is administered at a dose of about 3,360 mg.

In some embodiments, the antibody is administered at a dose of about 350 mg.

In some embodiments, the antibody is administered at a dose of about 750 mg.

In some embodiments, the antibody is administered at a dose of about 800 mg.

In some embodiments, the antibody is administered at a dose of about 850 mg.

In some embodiments, the antibody is administered at a dose of about 900 mg.

In some embodiments, the antibody is administered at a dose of about 950 mg.

In some embodiments, the antibody is administered at a dose of about 1,000 mg.

In some embodiments, the antibody is administered at a dose of about 1,050 mg.

In some embodiments, the antibody is administered at a dose of about 1,100 mg.

In some embodiments, the antibody is administered at a dose of about 1,150 mg.

In some embodiments, the antibody is administered at a dose of about 1,200 mg.

In some embodiments, the antibody is administered at a dose of about 1,250 mg.

In some embodiments, the antibody is administered at a dose of about 1,300 mg.

In some embodiments, the antibody is administered at a dose of about 1,350 mg.

In some embodiments, the antibody is administered at a dose of about 1,400 mg.

In some embodiments, the antibody is administered at a dose of about 1,600 mg.

In some embodiments, the antibody is administered at a dose of about 1,750 mg.

In some embodiments, the antibody is administered at a dose of about 2,240 mg.

In some embodiments, the antibody is administered at a dose of about 2,100 mg.

In some embodiments, the antibody is administered at a dose of about 2,400 mg.

In some embodiments, the antibody is administered at a dose of about 3,360 mg.

In certain embodiments, the antibody is administered at a dose of 1,050 mg for body weigh <80 kg and 1,400 mg for body weight ≥80 kg.

In particular embodiments, the antibody is administered at a dose of 700 mg for body weigh <80 kg and 1,050 mg for body weight ≥80 kg.

In particular embodiments, the antibody is administered at a dose of 1,750 mg for body weigh <80 kg and 2,100 mg for body weight ≥80 kg.

In certain embodiments, the antibody is administered at a dose of 1,600 mg for body weigh <80 kg and 2,240 mg for body weight ≥80 kg.

In certain embodiments, the antibody is administered at a dose of 2,400 mg for body weigh <80 kg and 3,360 mg for body weight ≥80 kg.

In some embodiments, the antibody is administered twice a week.

In certain embodiments, the antibody is administered once a week.

In some embodiments, the antibody is administered once every two weeks.

In certain embodiments, the antibody is administered once every three weeks.

In some embodiments, the antibody is administered once every four weeks.

In certain embodiments, the antibody is administered once a week or once every three weeks.

In particular embodiments, the antibody is administered once weekly for the first 3 weeks and then once every 3 weeks.

In some embodiments, the antibody is administered on a 21-day cycle.

In some embodiments, the subject has a body weight (BW) of <80 kg, and the antibody (e.g., bispecific antibody such as amivantamab) is administered at a dose of 1600 mg on Cycle 1 Day 1 and then at a dose of 2400 mg once every week (Q1W) for the remainder of Cycle 1 (Days 8 and 15), and once every 3 weeks (Q3W) from Cycle 2 onwards. In some embodiments, the antibody is administered subcutaneously.

In some embodiments, the subject has a body weight (BW) of 80 kg or larger, and the antibody (e.g., bispecific antibody such as amivantamab) is administered at a dose of 2240 mg on Cycle 1 Day 1 and then at a dose of 3360 mg once every week (Q1W) for the remainder of Cycle 1 (Days 8 and 15), and once every 3 weeks (Q3W) from Cycle 2 onwards. In some embodiments, the antibody is administered subcutaneously.

In some embodiments, the subject is further administered one or more anticancer agents. In some embodiments, one or more anticancer agents is an PD-(L)1 axis inhibitor. In some embodiments, the PD-(L)1 axis inhibitor is an antibody specific to PD-1. In some embodiments, the PD-(L)1 axis inhibitor is pembrolizumab (KEYTRUDA®).

In some embodiments, the subject has a body weight (BW) of <80 kg, and the antibody (e.g., bispecific antibody such as amivantamab) is administered at a dose of 1600 mg on Cycle 1 Day 1 and then at a dose of 2400 mg once every week (Q1W) for the remainder of Cycle 1 (Days 8 and 15), and once every 3 weeks (Q3W) from Cycle 2 onwards, along with intravenous injection of pembrolizumab 200 mg Q3W. In some embodiments pembrolizumab is administered on Day 1 of each 21-day cycle.

In some embodiments, the subject has a body weight (BW) of 80 kg or larger, and the antibody (e.g., bispecific antibody such as amivantamab) is administered at a dose of 2240 mg on Cycle 1 Day 1 and then at a dose of 3360 mg once every week (Q1W) for the remainder of Cycle 1 (Days 8 and 15), and once every 3 weeks (Q3W) from Cycle 2 onwards, along with intravenous injection of pembrolizumab 200 mg Q3W. In some embodiments pembrolizumab is administered on Day 1 of each 21-day cycle.

In some embodiments, the subject has a body weight (BW) of <80 kg, and the antibody (e.g., bispecific antibody such as amivantamab) is administered at a dose of 1600 mg on Cycle 1 Day 1 and then at a dose of 2400 mg once every week (Q1W) for the remainder of Cycle 1 (Days 8 and 15), and once every 3 weeks (Q3W) from Cycle 2 onwards, along with intravenous injection of paclitaxel 175 mg/m² Q3W (on Day 1 of each 21-day cycle).

In some embodiments, the subject has a body weight (BW) of 80 kg or larger, and the antibody (e.g., bispecific antibody such as amivantamab) is administered at a dose of 2240 mg on Cycle 1 Day 1 and then at a dose of 3360 mg once every week (Q1W) for the remainder of Cycle 1 (Days 8 and 15), and once every 3 weeks (Q3W) from Cycle 2 onwards, along with intravenous injection of paclitaxel 175 mg/m2 Q3W (on Day 1 of each 21-day cycle).

In some embodiments, the subject has a body weight (BW) of <80 kg, and the antibody (e.g., bispecific antibody such as amivantamab) is administered at a dose of 1600 mg on Cycle 1 Day 1 and then at a dose of 2400 mg once every week (Q1W) for the remainder of Cycle 1 (Days 8 and 15), and once every 3 weeks (Q3W) from Cycle 2 onwards, along with intravenous injection of pembrolizumab 200 mg Q3W (on Day 1 of each 21-day cycle) and intravenous injection of carboplatin AUC of 5 mg/mL/min Q3W (on Day 1 of each 21-day cycle). In some embodiments, on Day 1 of each cycle, pembrolizumab is administered first, followed by carboplatin (timing per the local guidelines and SmPC regulatory labeling) and amivantamab SC last. In some embodiments, amivantamab is administered at least 1 hour after carboplatin infusion.

In some embodiments, the subject has a body weight (BW) of 80 kg or larger, and the antibody (e.g., bispecific antibody such as amivantamab) is administered at a dose of 2240 mg on Cycle 1 Day 1 and then at a dose of 3360 mg once every week (Q1W) for the remainder of Cycle 1 (Days 8 and 15), and once every 3 weeks (Q3W) from Cycle 2 onwards, along with intravenous injection of pembrolizumab 200 mg Q3W (on Day 1 of each 21-day cycle) and intravenous injection of carboplatin AUC of 5 mg/mL/min Q3W (on Day 1 of each 21-day cycle). In some embodiments, on Day 1 of each cycle, pembrolizumab is administered first, followed by carboplatin (timing per the local guidelines and SmPC regulatory labeling) and amivantamab SC last. In some embodiments, amivantamab should be administered at least 1 hour after carboplatin infusion.

In some embodiments, the anti-EGFR/c-Met bispecific antibody is administered using a different dosing regimen, for example more frequent (Q1W or Q2W) or less frequent (Q4W), than described, wherein the subject's exposure is deemed equivalent. For example, the EGFR/c-Met antibody is used in accordance the FDA-approved drug label for RYBREVANT™ (BLA No. 761443 and BLA No. 761210), which can be found, for example, at https://www.accessdata.fda.gov/drugsatfda_docs/label/2025/761433s000lbl.pdf and https://www.accessdata.fda.gov/drugsatfda_docs/label/2025/761210s011lbl.pdf, and are hereby incorporated by reference in its entirety for all purposes.

Additional information regarding amivantamab can be found, for example, in the prescribing information product insert for RYBREVANT® (amivantamab-vmjw) (www.janssenlabels.com/package-insert/product-monograph/prescribing-information/RYBREVANT-pi.pdf), which is incorporated herein by reference.

Additional information regarding the use of amivantamab in patients can be found, for example, in Park K. et al., Amivantamab in EGFR Exon 20 Insertion-Mutated Non-Small-Cell Lung Cancer Progressing on Platinum Chemotherapy: Initial Results From the CHRYSALIS Phase I Study. J Clin Oncol. 2021 Oct. 20; 39 (30): 3391-3402; Vyse S, Huang P H. Amivantamab for the treatment of EGFR exon 20 insertion mutant non-small cell lung cancer. Expert Rev Anticancer Ther. 2021 Dec. 16; and Cho B C et al., MARIPOSA: phase 3 study of first-line amivantamab+lazertinib versus osimertinib in EGFR-mutant non-small cell lung cancer. Future Oncol. 2021 Dec. 16; which are incorporated herein by reference.

In some embodiments, the anti-EGFR/c-Met bispecific antibody is administered to a subject having a HNSCC cancer that is HPV positive. In some embodiments, the antibody is administered to a subject having a HNSCC cancer that is HPV unrelated. In some embodiments, the antibody is administered to a subject having a HNSCC cancer with unknown HPV infection status. In some embodiments, the antibody is administered to a subject having a cancer of the oropharynx. In some embodiments, the antibody is administered to a subject having a cancer of the oral cavity, oropharynx, pharynx, or larynx. In some embodiments, the antibody is administered to a subject having a cancer of the oropharynx that is HPV negative. In some embodiments, the antibody is administered to a subject having a cancer of the oropharynx that is HPV positive or HPV status unknown.

In some embodiments, the method achieves a partial response or complete response in the subject, as defined by investigator assessment using Response Criteria in Solid Tumors (RECIST) version 1.1.

In some embodiments, the subcutaneous composition comprises:
about 160 mg/mL of the bispecific antibody, wherein the HCl of the bispecific antibody comprises the amino acid sequence of SEQ ID NO: 17, the LC1 of the bispecific antibody comprises the amino acid sequence of SEQ ID NO: 18, the HC2 of the bispecific antibody comprises the amino acid sequence of SEQ ID NO: 19, and the LC2 of the bispecific antibody comprises the amino acid sequence of SEQ ID NO: 20,
about 30 mM acetate and/or pharmaceutically acceptable acetate salt,
about 8.5% sucrose,
about 1 mg/mL L-methionine
polysorbate 80 to a final concentration of about 0.06% (w/v)
EDTA to a final concentration of about 20 μg/mL,
rHuPH20 to a final concentration of about 2,000 U/mL, and
a pH of about 5.7,

In certain embodiments, the anti-EGFR/c-Met bispecific antibody is administered using a dosing regimen different, for example more frequent (Q1W or Q2W) or less frequent (Q4W), than described below based on weight wherein the subject's exposure is deemed equivalent.
a) In some embodiments, the subject has a body weight (BW) of <80 kg, and the antibody (e.g., bispecific antibody such as amivantamab) is administered at a dose of 1600 mg on Cycle 1 Day 1 and then at a dose of 2400 mg once every week (Q1W) for the remainder of Cycle 1 (Days 8 and 15), and once every 3 weeks (Q3W) from Cycle 2 onwards.
b) In some embodiments, the subject has a body weight (BW) of 80 kg or larger, and the antibody (e.g., bispecific antibody such as amivantamab) is administered at a dose of 2240 mg on Cycle 1 Day 1 and then at a dose of 3360 mg once every week (Q1W) for the remainder of Cycle 1 (Days 8 and 15), and once every 3 weeks (Q3W) from Cycle 2 onwards.

### Additional Therapeutic Agents

In some embodiments, the subject is further administered one or more anticancer agents. In some embodiments, the anti-EGFR/c-Met antibody (e.g., bispecific antibody) and the one or more additional therapeutic agents (e.g., chemotherapeutic agents) are administered according to each of their respective dosing schedules during the same treatment cycles. In some embodiments, the antibody and the one or more additional therapeutic agents are administered separately (e.g., sequentially). For combination therapies, the one or more anti-cancer agents may be administered using recommended doses and dosages of the anti-cancer agent.

In some embodiments, one or more anticancer agents is an PD-(L)1 axis inhibitor. In some embodiment, the PD-(L)1 inhibitor is an antibody. In some embodiments, the antibody binds to PD-1 or PD-L1, and includes nivolumab (OPDIVO®), pembrolizumab (KEYTRUDA®), sintilimab, cemiplimab (LIBTAYOR), tripolibamab, tislelizumab, spartalizumab, camrelizumab, dostralimab, genolimzumab or cetrelimab, envafolimab, atezolizumab (TECENTRIQ®), durvalumab (IMFINZI®), avelumab (BAVENCIOR), REGN2810, pidilizumab, MEDI0680, PDR001, PF-06801591, BGB-A317, TSR-042, and SHR-1210. In some embodiments, the PD-(L)1 axis inhibitor is an antibody specific to PD-1. In some embodiments, the PD-(L)1 axis inhibitor is pembrolizumab (KEYTRUDA®).

In some embodiments, the anti-PD-(L)1 axis antibody comprises a heavy chain complementarity determining region 1 (HCDR1) of SEQ ID NO: 21, a HCDR2 of SEQ ID NO: 22, a HCDR3 of SEQ ID NO: 23, a light chain complementarity determining region 1 (LCDR1) of SEQ ID NO: 24, a LCDR2 of SEQ ID NO: 25 and a LCDR3 of SEQ ID NO: 26. In some embodiments, the anti-PD-(L)1 axis antibody comprises a heavy chain variable domain (VH) of SEQ ID NO: 27 and a light chain variable domain (VL) of SEQ ID NO: 28. In some embodiments, the anti-PD-(L)1 axis antibody comprises a heavy chain (HC) of SEQ ID NO: 29 and a light chain (LC) of SEQ ID NO: 30.

In some embodiments, the anti-PD-(L)1 axis antibody is administered at a dose of 200 mg once in three weeks (Q3W). In some embodiments, the anti-PD-(L)1 axis antibody is administered on Day 1 of each 21-day cycle. In some embodiments, the anti-PD-(L)1 axis antibody is administered at a dose of 400 mg once in six weeks (Q6W). In some embodiments, the anti-PD-(L)1 axis antibody is administered intravenously. In some embodiments, the anti-PD-(L)1 axis antibody is administered subcutaneously. In some embodiments, the anti-PD-(L)1 axis antibody is administered per local practice guidelines.

In some embodiments, the anti-PD-(L)1 axis antibody is pembrolizumab and is administered at a dose of 200 mg once in three weeks (Q3W).). In some embodiments, pembrolizumab is administered on Day 1 of each 21-day cycle. In some embodiments, pembrolizumab is administered over a 30-minute infusion time.

In some embodiments, the anti-PD-(L)1 axis antibody is administered to a subject, wherein the subject's PD-L1 combined positive score (CPS) is determined. In some embodiments, the CPS indicates the PD-L1 protein levels in the tumor tissue. In some embodiments, the CPS is determined using immunohistochemistry. In some embodiments, the immunohistochemistry is performed using 22C3 antibody. In some embodiments, the CPS is determined using a companion diagnostic test for prembrolizumab.

In some embodiments, the anti-PD-(L)1 axis antibody is administered to a subject, wherein the subject has a PD-L1 combined positive score (CPS) greater than or equal to 1. In some embodiments, the anti-PD-(L)1 axis antibody is administered to a subject, wherein the subject has a PD-L1 combined positive score (CPS) of 1-19. In some embodiments, the anti-PD-(L)1 axis antibody is administered to a subject, wherein the subject has a PD-L1 combined positive score (CPS) greater than or equal to 20. In some embodiments, the anti-PD-(L)1 axis antibody is administered to a subject, wherein the subject has a PD-L1 combined positive score (CPS) less than 1.

### PD-(L)1 Axis Inhibitory Antibody Sequences

An exemplary anti-PD-(L)1 axis antibody that can be used in the methods of the disclosures is Pembrolizumab. Pembrolizumab is characterized by following amino acid sequences:

Formula (I)
>(HCDR1)
SEQ ID NO: 21
NYYMY
>(HCDR2)
SEQ ID NO: 22
GINPSNGGTNFNEKFKN
>(HCDR3)
SEQ ID NO: 23
RDYRFDMGFDY
>(LCDR1)
SEQ ID NO: 24
RASKGVSTSGYSYLH
>(LCDR2)
SEQ ID NO: 25
LASYLES
>(LCDR3)
SEQ ID NO: 26
QHSRDLPLT
>(VH)
SEQ ID NO: 27
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSS
>(VL)
SEQ ID NO: 28
EIVLTQSPATLSLSPGERATLSCRASKGVSTSGYSYLHWYQQKPGQAPR
LLIYLASYLESGVPARFSGSGSGTDFTLTISSLEPEDFAVYYCQHSRDL
PLTFGGGTKVEIK
>(HC)
SEQ ID NO: 29
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSASTKGPSVFPLAPCSRSTSESTAALGC
LVKDYFPEPVTVSWNSGALTSGVHTFPAVLQSSGLYSLSSVVTVPSSSL
GTKTYTCNVDHKPSNTKVDKRVESKYGPPCPPCPAPEFLGGPSVFLFPP
KPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSL
SLSLGK
>(LC)
SEQ ID NO: 30
EIVLTQSPATLSLSPGERATLSCRASKGVSTSGYSYLHWYQQKPGQAPR
LLIYLASYLESGVPARFSGSGSGTDFTLTISSLEPEDFAVYYCQHSRDL
PLTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

In certain embodiments, the method further comprises administering to the subject one or more additional therapeutic agents. Non-limiting examples of the one or more additional therapeutic agents include a T cell expressing chimeric antigen receptor (CAR) (CAR-T cell), a natural killer cell expressing CAR (CAR-NK cell), a macrophage expressing CAR (CAR-M cell), a chemotherapeutic agent, an immune checkpoint inhibitor, a T-cell redirector, radiation therapy, surgery and a standard of care drug. In certain embodiments, the one or more additional therapeutic agents comprises chemotherapy, radiation therapy, surgery, a targeted anti-cancer therapy, a kinase inhibitor, or a combination thereof.

In some embodiments, the one or more additional therapeutic agents are one or more anti-cancer therapies. In some embodiments, the one or more additional therapeutic agents comprise one or more chemotherapeutic agents.

### Platinum-Based Chemotherapy Agents

In some embodiments, one or more anticancer agents is a platinum-based chemotherapy agent. In some embodiments, the platinum-based chemotherapy agent is a standard of care platinum therapy. In some embodiments, the platinum-based chemotherapy agent is carboplatin or cisplatin. In some embodiments, the platinum-based chemotherapy agent is carboplatin. In some embodiments, the platinum-based chemotherapy agent is cisplatin.

An exemplary platinum-based chemotherapy agent that can be used in the methods of the disclosures is Carboplatin. In some embodiments, the platinum-based chemotherapy agent is platinum, diammine [1,1-cyclobutane-dicarboxylato (2-)-0,0′]-, (SP-4-2). In some embodiments, the platinum-based chemotherapy agent has a structure of Formula (II):

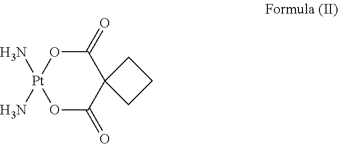

In some embodiments, carboplatin is administered for a target AUC of 5 mg/mL/min as an IV infusion on Cycle Day 1, Q3W. In some embodiments, the maximum carboplatin dose should not exceed the target AUC (mg*min/mL)*150 mL/min, but it may be less. In some embodiments, the maximum dose cannot exceed a total dose of 750 mg. In some embodiments, carboplatin is administered over 15 to 60-min infusion time. In some embodiments, carboplatin is administered for 6 cycles. In some embodiments, carboplatin is administered for more than 6 cycles. In some embodiments, carboplatin is administered per local practice guidelines.

### Subjects

The terms “subject” and “patient” can be used interchangeably herein. “Patient in need thereof” or “subject in need thereof” refers to a mammalian subject, preferably human, diagnosed with or suspected of having a disease, to whom will be or has been administered a bi-specific anti-EGFR anti-MET antibody according to a method of the invention. “Patient in need thereof” or “subject in need thereof” includes those subjects already with the undesired physiological change or disease as well as those subjects prone to have the physiological change or disease.

In some embodiments, the subject is 18 years of age or older, e.g., 18 to less than 40 years of age, 18 to less than 45 years of age, 18 to less than 50 years of age, 18 to less than 55 years of age, 18 to less than 60 years of age, 18 to less than 65 years of age, 18 to less than 70 years of age, 18 to less than 75 years of age, 40 to less than 75 years of age, 45 to less than 75 years of age, 50 to less than 75 years of age, 55 to less than 75 years of age, 60 to less than 75 years of age, 65 to less than 75 years of age, 60 to less than 75 years of age, 40 years of age or older, 45 years of age or older, 50 years of age or older, 55 years of age or older, 60 years of age or older, 65 years of age or older, 70 years of age or older or 75 years of age or older.

In some embodiments, the subject is a child. In some embodiments, the subject is 18 years of age or younger, e.g., 0-18 years of age, 0-12 years of age, 0-16 years of age, 0-17 years of age, 2-12 years of age, 2-16 years of age, 2-17 years of age, 2-18 years of age, 3-12 years of age, 3-16 years of age, 3-17 years of age, 3-18 years of age, 4-12 years of age, 4-16 years of age, 4-17 years of age, 4-18 years of age, 6-12 years of age, 6-16 years of age, 6-17 years of age, 6-18 years of age, 9-12 years of age, 9-16 years of age, 9-17 years of age, 9-18 years of age, 12-16 years of age, 12-17 years of age or 12-18 years of age.

In some embodiments, the subject has been diagnosed with head and neck squamous cell carcinoma (HNSCC) for at least about 1 month, e.g., at least about: 2 months, 3 months, 4 months, 5 months, 6 months, 7 months, 8 months, 9 months, 10 months, 11 months, 1 year, 18 months, 2 years, 30 months, 3 years, 4 years, 5 years, 6 years, 7 years, 8 years, 9 years or 10 years. In particular embodiments, the subject is newly diagnosed with HNSCC.

In some embodiments, the HNSCC is recurrent and/or metastatic. In some embodiments, the HNSCC is recurrent. In some embodiments, the HNSCC is metastatic.

In some embodiments, the HNSCC is unresectable.

In some embodiments, the subject has EGFR or c-Met expressing cancer.

In some embodiments, the HNSCC is HNSCC of the oropharynx, oral cavity, hypopharynx, pharynx, or larynx. In some embodiments, the HNSCC is HPV infection negative. In some embodiments, the HNSCC is HPV infection negative, identified by negative p16 status or high-risk HPV in-situ hybridization (ISH) in tissue.

In some embodiments, the HNSCC is HPV infection positive. In some embodiments, the HNSCC is HPV infection positive, identified by positive p16 status, HPV DNA test, or high-risk HPV in-situ hybridization (ISH) in tissue. In some embodiments, the HNSCC is HPV infection positive, identified by positive p16 status. In some embodiments, the HNSCC is HPV infection positive, identified by positive HPV DNA test. In some embodiments, the HNSCC is HPV infection positive, identified by HPV in-situ hybridization (ISH) in tissue. In some embodiments, tissue is tumor tissue. In some embodiments, the HNSCC is HNSCC of the oropharynx, oral cavity, hypopharynx, pharynx or larynx, and HPV infection positive.

In some embodiments, the HNSCC is HNSCC of the oropharynx, oral cavity, hypopharynx, pharynx, or larynx. In some embodiments, the HNSCC is HPV infection status is unknown.

In some embodiments, the HNSCC is HNSCC of the oropharynx, oral cavity, hypopharynx, pharynx, or larynx. In some embodiments, the HNSCC is HPV infection status is negative. In some embodiments, the HNSCC is HNSCC of the oropharynx and is HPV negative.

Presence of HPV infection in tissue can be determined by various methods known in the art. In some embodiments, the presence of HPV infection is determined by DNA in situ hybridization (ISH), RT-PCR for E6 and E7 mRNA, RNA ISH, or p16 immunohistochemistry (IHC). In some embodiments, the presence of HPV infection is determined by p16 immunohistochemistry (IHC). In some embodiments, the presence of HPV infection may be determined as described in James S. Lewis Jr. p16 immunohistochemistry As a Standalone Test for Risk Stratification in Oropharyngeal Squamous Cell Carcinoma. Head and Neck Pathol (2012) 6: S75-S82, which is incorporated herein by reference in its entirety.

In certain embodiments, the subject is treatment naïve. In some embodiments, the subject is treatment naïve in the recurrent or metastatic setting. In some embodiments, the subject is treatment naïve for systemic therapy. In some embodiments, the subject has received curative systemic therapy for locally advanced disease more than 6 months prior to enrollment. In some embodiments, the subject received anti-PD-1/PD-L1/PD-L2 agents more than 12 months prior to enrollment.

In some embodiments, the subject has received one or more prior anti-cancer therapies. In certain embodiments, the one or more prior anti-cancer therapies comprises one or more chemotherapeutic agents, checkpoint inhibitors, targeted anti-cancer therapies or kinase inhibitors, or any combination thereof. In particular embodiments, the subject is relapsed or resistant to treatment with one or more prior anti-cancer therapies.

In some embodiments, the subject has received prior treatment with platinum-based chemotherapy and a PD-1/PD-L1 inhibitor. In some embodiments, the subject has received prior treatment with platinum-based chemotherapy. In some embodiments, the subject has received prior treatment with a PD-1/PD-L1/PD-L2 inhibitor. In some embodiments, the subject has received prior treatment with the PD-1/PD-L1/PD-L2 inhibitor more than 12 months prior to receiving the treatment of the invention.

In some embodiments, the subject has not previously received anti-EGFR therapy. In some embodiments, the subject has not previously received anti-EGFR antibody therapy. In some embodiments, the subject has not previously received anti-EGFR tyrosine kinase inhibitor (TKI) therapy.

In some embodiments, a method of treating recurrent or metastatic head and neck squamous cell carcinoma (HNSCC) in a subject in need thereof comprises administering a therapeutically effective amount of the anti-EGFR/anti-cMet antibody (e.g., amivantamab) as a monotherapy, wherein the subject has received prior treatment with platinum-based chemotherapy and a PD-1/PD-L1 inhibitor, wherein the antibody is administered in 21-day cycles (i) in an amount of 1,600 mg if the subject has a body weight of less than 80 kg (or an amount of 2,240 mg if the subject has a body weight of ≥80 kg) on Cycle 1 Day 1, and then (ii) in an amount of 2,400 mg if the subject has a body weight of less than 80 kg (or an amount of 3,360 mg if the subject has a body weight of ≥80 kg) once weekly (Q1W) for the remainder of Cycle 1 (Days 8 and 15) and once every 3 weeks (Q3W) from Cycle 2 onwards. In some embodiments, the HNSCC is HPV negative. In some embodiments, HNSCC is HPV positive. In some embodiments, the HNSCC HPV status is unknown.

In some embodiments, a method of treating head and neck cancer in a subject in need thereof comprises administering a therapeutically effective amount of the anti-EGFR/anti-cMet antibody (e.g., amivantamab), pembrolizumab, and carboplatin, wherein the subject is treatment-naïve in a recurrent/metastatic head and neck cancer setting, wherein the anti-EGFR/c-Met antibody is administered in 21-day cycles (i) in an amount of 1,600 mg if the subject has a body weight of less than 80 kg (or an amount of 2,240 mg if the subject has a body weight of ≥80 kg) on Cycle 1 Day 1, and then (ii) in an amount of 2,400 mg if the subject has a body weight of less than 80 kg (or an amount of 3,360 mg if the subject has a body weight of ≥80 kg) once weekly (Q1W) for the remainder of Cycle 1 (Days 8 and 15) and once every 3 weeks (Q3W) from Cycle 2 onwards, and wherein the pembrolizumab is administered at a dose of 200 mg Q3W (on Day 1 of each 21-day cycle) starting in Cycle 1 (e.g., as an intravenous injection), and wherein the carboplatin is administered at a dose of AUC 5 mg/mL·min Q3W (on Day 1 of each 21-day cycle) starting in Cycle 1 (e.g., as an intravenous injection). In some embodiments, the carboplatin is administered on cycles 1-6. In some embodiments, pembrolizumab is administered first, followed by carboplatin, followed by amivantamab. In some embodiments, amivantamab is administered at least 1 hour after carboplatin infusion. In some embodiments, the HNSCC is HPV negative. In some embodiments, HNSCC is HPV positive. In some embodiments, the HNSCC HPV status is unknown.

In some embodiments, a method of treating head and neck cancer in a subject in need thereof comprises administering a therapeutically effective amount of the anti-EGFR/anti-cMet antibody (e.g., amivantamab) and paclitaxel, wherein the subject has received prior treatment with a PD-1/PD-L1 inhibitor, wherein the antibody is administered in 21-day cycles (i) in an amount of 1,600 mg if the subject has a body weight of less than 80 kg (or an amount of 2,240 mg if the subject has a body weight of ≥80 kg) on Cycle 1 Day 1, and then (ii) in an amount of 2,400 mg if the subject has a body weight of less than 80 kg (or an amount of 3,360 mg if the subject has a body weight of ≥80 kg) once weekly (Q1W) for the remainder of Cycle 1 (Days 8 and 15) and once every 3 weeks (Q3W) from Cycle 2 onwards, and wherein the paclitaxel is administered at a dose of 175 mg/m² Q3W (on Day 1 of each 21-day cycle) starting in Cycle 1 (e.g., as a solvent-based infusion of paclitaxel). In some embodiments, the HNSCC is HPV negative. In some embodiments, HNSCC is HPV positive. In some embodiments, the HNSCC HPV status is unknown.

In some embodiments, a method of treating head and neck cancer in a subject in need thereof comprises administering a therapeutically effective amount of the anti-EGFR/anti-cMet antibody (e.g., amivantamab) and paclitaxel, wherein the subject has received prior treatment with a PD-1/PD-L1 inhibitor, wherein the antibody is administered in 21-day cycles (i) in an amount of 1,050 mg if the subject has a body weight of less than 80 kg (or an amount of 1,400 mg if the subject has a body weight of ≥80 kg) on Cycle 1 Day 1, and then (ii) in an amount of 1,600 mg if the subject has a body weight of less than 80 kg (or an amount of 2,240 mg if the subject has a body weight of ≥80 kg) once weekly (Q1W) for the remainder of Cycle 1 (Days 8 and 15) and once every 3 weeks (Q3W) from Cycle 2 onwards, and wherein the paclitaxel is administered at a dose of 175 mg/m² Q3W (on Day 1 of each 21-day cycle) starting in Cycle 1 (e.g., as a solvent-based infusion of paclitaxel). In some embodiments, the HNSCC is HPV negative. In some embodiments, HNSCC is HPV positive. In some embodiments, the HNSCC HPV status is unknown.

In some embodiments, a method of treating recurrent or metastatic head and neck squamous cell carcinoma in a subject in need thereof comprises administering a therapeutically effective amount of the anti-EGFR/anti-cMet antibody (e.g., amivantamab) as a monotherapy, wherein the subject is HPV-positive and has received prior treatment with platinum-based chemotherapy and a PD-1/PD-L1 inhibitor, wherein the antibody is administered in 21-day cycles (i) in an amount of 1,600 mg if the subject has a body weight of less than 80 kg (or an amount of 2,240 mg if the subject has a body weight of ≥80 kg) on Cycle 1 Day 1, and then (ii) in an amount of 2,400 mg if the subject has a body weight of less than 80 kg (or an amount of 3,360 mg if the subject has a body weight of ≥80 kg) once weekly (Q1W) for the remainder of Cycle 1 (Days 8 and 15) and once every 3 weeks (Q3W) from Cycle 2 onwards.

In some embodiments of the separate methods described herein, the subject has one or more unresectable tumors that are treated. In further embodiments, one or more unresectable tumors are treated wherein one or more of the unresectable tumors are rendered resectable.

### EMBODIMENTS

Provided below are enumerated embodiments of the present invention. These embodiments are illustrative only and do not limit the scope of the present disclosure or of the claims attached hereto.
1. A method of treating head and neck cancer in a subject in need thereof, comprising administering to the subject a combination therapy, wherein the combination therapy comprises
   a therapeutically effective amount of an anti-epidermal growth factor receptor (EGFR)/hepatocyte growth factor receptor (c-Met) antibody;
   a therapeutically effective amount of pembrolizumab; and
   a therapeutically effective amount of carboplatin.
2. The method of embodiment 1, wherein the anti-EGFR/c-Met antibody comprises:
   a) a first domain that specifically binds EGFR, comprising heavy chain complementarity determining region 1 (HCDR1), HCDR2, HCDR3, light chain complementarity determining region 1 (LCDR1), LCDR2 and LCDR3 amino acid sequences of SEQ ID NOs: 1, 2, 3, 4, 5 and 6, respectively; and
   b) a second domain that specifically binds c-Met, comprising HCDR1, HCDR2, HCDR3, LCDR1, LCDR2 and LCDR3 amino acid sequences of SEQ ID NOs: 7, 8, 9, 10, 11 and 12, respectively.
3. The method of embodiment 2, wherein the first domain comprises a heavy chain variable region (VH) of SEQ ID NO:13 and a light chain variable region (VL) of SEQ ID NO:14, and the second domain comprises a VH of SEQ ID NO:15 and a VL of SEQ ID NO:16.
4. The method of any one of embodiments 1-3, wherein the antibody is of the IgG1 isotype.
5. The method of any one of embodiments 1-4, wherein the antibody comprises a first heavy chain (HCl) of SEQ ID NO:17, a first light chain (LC1) of SEQ ID NO:18, a second heavy chain (HC2) of SEQ ID NO:19 and a second light chain (LC2) of SEQ ID NO:20.
6. The method of any one of embodiments 1-5, wherein the antibody is an isolated bispecific antibody.
7. The method of embodiment 6, wherein the bispecific antibody is amivantamab.
8. The method of any one of embodiments 1-7, wherein the antibody comprises a biantennary glycan structure with a fucose content of about 1% to about 15% or less than about 20%.
9. The method of any one of embodiments 1-8, wherein the antibody is administered at a dose of about 1,600 mg to about 3,360 mg.
10. The method of embodiment 9, wherein the antibody is administered at a dose of about 1,600 mg, about 2,240 mg, about 2,400 mg, or about 3,360 mg.
11. The method of embodiment 10, wherein the antibody is administered at a dose of about 1,600 mg.
12. The method of embodiment 10, wherein the antibody is administered at a dose of about 2,240 mg.
13. The method of embodiment 10, wherein the antibody is administered at a dose of about 2,400 mg.
14. The method of embodiment 10, wherein the antibody is administered at a dose of about 3,360 mg.
15. The method of any one of embodiments 1-14, wherein the antibody is administered once a week or once every three weeks.
16. The method of embodiment 15, wherein the antibody is administered once weekly for the first 3 weeks and once every 3 weeks thereafter.
17. The method of any one of embodiments 1-16, wherein the antibody is administered at a dose of about 1,600 mg (2240 mg, if body weight is equal or larger than 80 kg) on Cycle 1 Day 1 and 2400 mg (3360 mg, if body weight is equal or larger than 80 kg) once every week (Q1W) for the remainder of Cycle 1 (Days 8 and 15), and every 3 weeks (Q3W) from Cycle 2 onwards, wherein each Cycle is 21 days.
18. The method of any one of embodiments 1-17, wherein pembrolizumab is administered at a dose of about 200 mg once every three weeks.
19. The method of embodiment 18, wherein pembrolizumab is administered on Day 1 of each 21-day cycle.
20. The method of any one of embodiments 1-19, wherein carboplatin is administered at a dose of about AUC 5 mg/mL·min once every three weeks.
21. The method of embodiment 20, wherein carboplatin is administered on Day 1 of each 21-day cycle.
22. The method of embodiment 21, wherein carboplatin is administered on Cycles 1-6.
23. The method of any one of embodiments 1-22, wherein the head and neck cancer is a head and neck squamous cell carcinoma (HNSCC).
24. The method of any one of embodiments 1-23, wherein the HNSCC is cancer of oropharynx, oral cavity, hypopharynx, or larynx.
25. The method of any one of embodiments 1-24, wherein the HNSCC is HPV negative.
26. The method of any one of embodiments 1-24, wherein the HNSCC is HPV positive.
27. The method of any one of embodiments 1-24, wherein the HNSCC HPV infection status is unknown.
28. The method of any one of embodiments 1-27, wherein the subject is treatment naïve.
29 The method of embodiment 28, wherein the subject is treatment naïve in the recurrent or metastatic setting.
30. The method of any one of embodiments 1-27, wherein the subject was administered at least one anti-cancer therapy previously.
31. The method of embodiment 30, wherein the at least one anti-cancer therapy is a curative intent therapy for locally advanced disease.
32. The method of embodiment 30, wherein the at least one anti-cancer therapy is not anti-EGFR or anti-PD-1/PD-L1 therapy.
33. The method of any one of embodiments 1-21, wherein the subject is 18 years of age or older.
34. A method of treating recurrent or metastatic head and neck squamous cell carcinoma in a subject in need thereof comprising administering a therapeutically effective amount of the anti-EGFR/anti-cMet antibody (e.g., amivantamab), pembrolizumab, and carboplatin, wherein the subject is treatment-naïve in a recurrent/metastatic head and neck cancer setting, wherein the anti-EGFR/anti-cMet antibody is administered in 21-day cycles (i) in an amount of 1,600 mg if the subject has a body weight of less than 80 kg (or an amount of 2,240 mg if the subject has a body weight of ≥80 kg) on Cycle 1 Day 1, and then (ii) in an amount of 2,400 mg if the subject has a body weight of less than 80 kg (or an amount of 3,360 mg if the subject has a body weight of ≥80 kg) once weekly (Q1W) for the remainder of Cycle 1 (Days 8 and 15) and once every 3 weeks (Q3W) from Cycle 2 onwards, and wherein the pembrolizumab is administered at a dose of 200 mg Q3W (on Day 1 of each 21-day cycle) starting in Cycle 1 (e.g., as an intravenous injection), and wherein the carboplatin is administered at a dose of AUC 5 mg/mL·min Q3W (on Day 1 of each 21-day cycle) starting in Cycle 1 (e.g., as an intravenous injection).
35. The method of embodiment 34, wherein the carboplatin is administered on Cycles 1-6.
36. The method of any one of embodiments 1-35, wherein the anti-EGFR/anti-cMet antibody is administered subcutaneously.
37. The method of any one of embodiments 1-35, wherein the anti-EGFR/anti-cMet antibody is administered intravenously.
38. The method of any one of embodiments 1-37, wherein the method achieves a partial response or complete response in the subject, as defined by investigator assessment using Response Criteria in Solid Tumors (RECIST) version 1.1.
39. The method of any one of embodiments 1-38, wherein the subject has a PD-L1 combined positive score (CPS) of greater than or equal to 1, between 1-19, greater than or equal to 20, or less than 1.
40. The method of any one of embodiments 1-39, wherein the subject has a CPS of greater than or equal to 1.
41. The method of any one of embodiments 1-39, wherein the subject has a CPS of 1-19.
42. The method of any one of embodiments 1-39, wherein the subject has a CPS of greater than or equal to 20.
43. The method of any one of embodiments 1-39, wherein the subject has a CPS of less than 1.
44. The method of any one of embodiments 1-43, wherein the method demonstrates improved Objective Response Rate (ORR) compared to 5-FU+pembrolizumab+platinum (carboplatin/cisplatin) therapy as described herein, for example in Example 3-Table 5.
45. The method of any one of embodiments 1-43, wherein the method demonstrates improved Overall Survival (OS) compared to 5-FU+pembrolizumab+platinum (carboplatin/cisplatin) therapy as described herein, for example in Example 3-Table 5.
46. The method of any one of embodiments 1-43, wherein the method demonstrates improved Progression Free Survival (PFS) compared to 5-FU+pembrolizumab+platinum (carboplatin/cisplatin) therapy as described herein, for example in Example 3-Table 5.
47. The method of any one of embodiments 1-43, wherein the method demonstrates improved Duration of Response (DoR) compared to 5-FU+pembrolizumab+platinum (carboplatin/cisplatin) therapy as described herein, for example in Example 3-Table 5.
48. The method of any one of embodiments 1-43, wherein the method demonstrates improved Clinical Benefit Rate (CBR) compared to 5-FU+pembrolizumab+platinum (carboplatin/cisplatin) therapy as described herein, for example in Example 3-Table 5.
49. The method of any one of embodiments 1-17, wherein the antibody is administered at a dose of about 1,600 mg on Cycle 1 Day 1 and about 2400 mg once every week (Q1W) for the remainder of Cycle 1 (Days 8 and 15), and once every 3 weeks (Q3W) from Cycle 2 onwards, wherein each Cycle is 21 days, if body weight is less than 80 kg.
50. The method of any one of embodiments 1-14, wherein the antibody is administered at a dose of about 2240 mg on Cycle 1 Day 1 and about 3360 mg once every week (Q1W) for the remainder of Cycle 1 (Days 8 and 15), and once every 3 weeks (Q3W) from Cycle 2 onwards, wherein each Cycle is 21 days, if body weight is equal or larger than 80 kg.
51. The method of any one of embodiments 1-50, wherein the head and neck cancer is HPV-unrelated.
52. The method of any one of embodiments 1-51, wherein pembrolizumab is administered for up to 35 cycles.
53. The method of any one of embodiments 1-51, wherein carboplatin is administered for up to 6 cycles.
54. The method of any one of embodiments 1-53, wherein the head and neck cancer is unresectable.

### Example 1. a Study of Amivantamab Alone or in Addition to Other Treatment Agents in Participants with Recurrent/Metastatic Head and Neck Cancer (OrigAMI-4)

The purpose of this study is to determine safety and preliminary efficacy of amivantamab monotherapy, amivantamab in addition to pembrolizumab, and amivantamab in addition to paclitaxel. The study will also confirm the recommended Phase 2 combination dose (RP2CD) for amivantamab in addition to paclitaxel. Details of the study are described in Table 1.

TABLE 1
Amivantamab in Patients with Recurrent/Metastatic Head and Neck Cancer.

| | |
|---|---|
| Protocol | 61186372HNC2002 |
| Number | |
| Protocol | A Phase 1b/2, Open-label Study of Amivantamab Monotherapy and Amivantamab |
| Title | in Addition to Standard of Care Therapeutic Agents in Participants with |
| | Recurrent/Metastatic Head and Neck Squamous Cell Carcinoma. |
| Amivanta | Amivantamab is a low-fucose, human, immunoglobulin G1-based bispecific |
| mab | antibody, with one antigen-binding fragment arm that binds EGFR and the other Fab |
| | arm that binds MET. By inhibiting epidermal growth factor receptor (EGFR) and |
| | mesenchymal epithelial transition factor (MET) signaling functions, either through |
| | blocking of ligand-induced activation and/or inducing receptor degradation, |
| | amivantamab disrupts these signaling pathways and prevents tumor growth and |
| | progression. Furthermore, binding of amivantamab to EGFR and MET on the |
| | surface of tumor cells can target them for destruction by immune effector cells, |
| | through Ab-dependent cellular cytotoxicity (ADCC) and Ab-dependent cellular |
| | trogocytosis (ADCT) mechanisms. Amivantamab targets tumors with activating and |
| | resistance EGFR mutations and MET mutations and amplifications. |
| | The US FDA granted amivantamab accelerated approval for the treatment of adult |
| | patients with locally advanced or metastatic NSCLC with EGFR exon 20 insertion |
| | mutations, as detected by an FDA-approved test, whose disease has progressed on or |
| | after platinum-based chemotherapy. Amivantamab is currently approved in multiple |
| | countries and marketed under the name RYBREVANT ®. |
| Study | This is an open-label, multicenter Ph1b/2 study of amivantamab (subcutaneous |
| Design | administration) as monotherapy and in addition to paclitaxel, pembrolizumab, or |
| and Drug | carboplatin in participants with recurrent or metastatic (R/M) HNSCC. |
| Admin- | Cohort 1: Amivantamab Monotherapy (Dose Expansion) |
| istration | Participants will receive subcutaneous injection of amivantamab monotherapy 1600 |
| | milligrams (mg) (2240 mg, if body weight >=80 kilograms [kg]) on Cycle 1 Day 1 |
| | and 2400 mg (3360 mg, if body weight >=80 kg) once every week (qlw) for the |
| | remainder of Cycle 1 (Days 8 and 15), and every 3 weeks (q3w) from Cycle 2 |
| | onwards. |
| | Cohort 2: Amivantamab + Pembrolizumab (Dose Expansion Including Safety Run- |
| | in) |
| | Participants will receive subcutaneous injection of amivantamab 1600 mg (2240 mg, |
| | if body weight >=80 kg) on Cycle 1 Day 1 and 2400 mg (3360 mg, if body weight |
| | >=80 kg) qlw for the remainder of Cycle 1 (Days 8 and 15), and q3w from Cycle 2 |
| | onwards, along with intravenous injection of pembrolizumab 200 mg q3w (on Day 1 |
| | of each 21-day cycle). |
| | Cohort 3A (Dose Confirmation): Amivantamab + Paclitaxel |
| | Participants will receive subcutaneous injection of amivantamab 1600 mg (2240 mg, |
| | if body weight >=80 kg) on Cycle 1 Day 1 and 2400 mg (3360 mg, if body weight |
| | >=80 kg) qlw for the remainder of Cycle 1 (Days 8 and 15), and q3w from Cycle 2 |
| | onwards, along with intravenous injection of paclitaxel 175 mg/m\hat{ }2 q3w (on Day 1 |
| | of each 21-day cycle) in dose confirmation Cohort 3A. The recommended Phase 2 |
| | combination dose (RP2CD) of amivantamab will be determined in conjunction with |
| | study evaluation team (SET) in this dose confirmation Cohort 3A. |
| | Cohort 3B (Dose Expansion): Amivantamab + Paclitaxel |
| | Participants will receive subcutaneous injection of amivantamab at the determined |
| | RP2CD in addition to intravenous injection of paclitaxel 175 mg/m\hat{ }2 q3w (on Day 1 |
| | of each 21-day cycle) as confirmed by SET in Cohort 3A. |
| | Cohort 4: Amivantamab Monotherapy |
| | Participants will receive subcutaneous injection of amivantamab monotherapy 1600 |
| | mg (2240 mg, if body weight >=80 kg) on Cycle 1 Day 1 and 2400 mg (3360 mg, if |
| | body weight >=80 kg) q1w for the remainder of Cycle 1 (Days 8 and 15), and q3w |
| | from Cycle 2 onwards. |
| | Cohort 5: Pembrolizumab + Amivantamab + Carboplatin |
| | Participants will receive subcutaneous injection of amivantamab 1600 mg (2240 mg, |
| | if body weight >=80 kilograms [kg]) on Cycle 1 Day 1 and 2400 mg (3360 mg, if body |
| | weight >=80 kg) once every week (qlw) for the remainder of Cycle 1 (Days 8 and |
| | 15), and every 3 weeks (q3w) from Cycle 2 onwards, along with intravenous injection |
| | of pembrolizumab 200 mg q3w (on Day 1 of each 21-day cycle), and intravenous |
| | injection of carboplatin AUC 5 mg/mL.minc Q3W (on Day 1 of Cycles 1-6). |
| Key | 1. Have histologically or cytologically confirmed recurrent/metastatic head and |
| Inclusion | neck squamous cell carcinoma (R/M HNSCC) that is considered incurable by |
| Criteria | local therapies. Acceptable prior lines of therapy will be determined according to |
| | specific cohort 1, 2, 3A and 3B: (a) The eligible primary tumor locations are the |
| | oropharynx, oral cavity, hypopharynx, or larynx; (b) Any known p16 status of |
| | tumor must be negative (Note: All participants with an oropharyngeal tumor |
| | must have results of p16 status, per local testing); (c) Participants must provide |
| | local testing results of programmed cell death ligand 1 (PD-L1) status, if |
| | available; Cohort 4: (a) Patients must have primary tumor location in |
| | oropharynx. Unknown primary tumors are not included (b) Primary tumor must |
| | be HPV-positive, confirmed by positive p16 test or high-risk human |
| | papillomavirus (HPV) in-situ hybridization (ISH) in tissue (current or archival) |
| | (c) Participants must provide local testing results of PD-L1 status, if available |
| | 2. Participants in Cohorts 1, 2, 3B, and 4 must have measurable disease according |
| | to RECIST version 1.1 (Eisenhauer et al. EUROPEAN JOURNAL OF |
| | CANCER 45 (2009) 228-247). Participants in Cohort 3A must have evaluable |
| | disease (defined as having at least 1 non-target lesion according to RECIST |
| | version 1.1 |
| | 3. Toxicities from previous anticancer therapies should have resolved to baseline |
| | levels or to Grade 1 or less prior to the first dose of study treatment (except for |
| | alopecia or post-radiation skin changes [any grade], Grade less than or equal to |
| | [<=]2 peripheral neuropathy and Grade <=2 hypothyroidism stable on hormone |
| | replacement) |
| | 4. Have an Eastern Cooperative Oncology Group (ECOG) performance status of 0 |
| | to 1 |
| | 5. Participant must have adequate organ and bone marrow function as follows, |
| | without history of red blood cell transfusion, platelet transfusion, or use of |
| | granulocyte colony-stimulating factor within 7 days prior to the date of the |
| | laboratory test. |
| | 6. Participants should have: a) Hemoglobin >=9 grams per deciliter (g/dL); b) |
| | Neutrophils >=1.5 x 10³/mcg; c) Platelets >=100 x 10³/mcg |
| | 7. Participants in Cohort 5: |
| | a. Have histologically or cytologically confirmed R/M HNSCC that is |
| | considered incurable by local therapies. |
| | b. The eligible primary tumor locations are the oropharynx, oral cavity, |
| | hypopharynx, or larynx. |
| | c. HPV status must be known (either positive or negative) for patients with |
| | primary tumor location in oropharynx with p16 test or high-risk HPV ISH |
| | in tissue. |
| | d. Participants must provide local testing results of PD-L1 status. |
| | e. Have measurable disease according to RECIST v1.1. |
| | f. Participants must have documented local testing results demonstrating a PD- |
| | L1 CPS ≥1 (using a 22C3 antibody test) within 6 months of the first dose of |
| | study treatment. |
| | g. Participant must be treatment-naive in the recurrent and/or metastatic |
| | setting. Systemic therapy which was completed more than 6 months prior to |
| | first study treatment administration, if given as part of treatment for locally |
| | advanced disease with curative intent, is allowed except for anti-EGFR or |
| | anti-PD-1/PD-L1 therapy |
| Key | 1. Uncontrolled illness including any medical history or current (non-infectious) |
| Exclusion | interstitial lung disease (ILD)/ pneumonitis/ pulmonary fibrosis, or where |
| Criteria | suspected ILD/pneumonitis/pulmonary fibrosis cannot be ruled out by imaging |
| | at screening |
| | 2. Participant with untreated brain metastases leptomeningeal disease, or spinal |
| | cord compression not definitively treated with surgery or radiation |
| | 3. Participant with a history of clinically significant cardiovascular disease |
| | 4. Received prior chemotherapy, targeted cancer therapy, immunotherapy, or |
| | treatment with an investigational anticancer agent within 2 weeks or 4 half-lives, |
| | whichever is longer, before the first administration of study treatment. The |
| | maximum required washout is 28 days |
| | 5. Received radiotherapy for palliative purposes within 7 days of the first |
| | administration of study treatment |
| Key | Primary Outcome Measures: |
| Study | Cohorts 1, 2, 3B and 4: Objective Response Rate (ORR) [Time Frame: 2 |
| Measures | years and 2 months] |
| | ORR is defined as the proportion of participants who achieve either a partial |
| | response (PR) or complete response (CR), as defined by investigator assessment |
| | using Response Criteria in Solid Tumors (RECIST) version 1.1. |
| | Cohort 3A: Number of Participants With Dose-limiting Toxicities (DLT) [ |
| | Time Frame: Up to 21 days] |
| | Number of participants with DLTs will be reported. A DLT is defined as any of the |
| | following: treatment delay of greater than (>) 28 days due to unresolved toxicity, |
| | non-hematologic toxicity of Grade 3 or higher, hematologic toxicity of Grade 4 |
| | neutropenia persisting for >7 days or Grade >3 thrombocytopenia with clinically |
| | significant bleeding or neutropenic fever of any grade, and liver enzyme elevation. |
| | Cohort 3A: Number of Participants With Treatment-emergent Adverse |
| | Events (TEAEs) as a Measure of Severity [Time Frame: 2 years and 1 month] |
| | An AE is any untoward medical occurrence in a participant participating in a clinical |
| | study that does not necessarily have a causal relationship with the pharmaceutical/ |
| | biological agent under study. TEAEs are defined as AEs with onset or worsening on |
| | or after date of first dose of study treatment. Severity of TEAEs will be graded |
| | according to the National Cancer Institute Common Terminology Criteria for |
| | Adverse Events (NCI-CTCAE) version 5.0. Severity scale ranges from Grade 1 |
| | (mild) to Grade 5 (death). Grade 1 = mild, Grade 2 = moderate, Grade 3 = severe, |
| | Grade 4 = life-threatening, and Grade 5 = death related to adverse event. |
| | Cohort 5: the primary endpoint is ORR, defined as the proportion of |
| | participants who achieve either a PR or CR, as defined by investigator assessment |
| | using RECIST v1.1. The ORR and its exact 95% CI will be calculated for the |
| | response-evaluable population descriptively for each cohort separately. A Fisher's |
| | exact test will be used to compare the ORR with the threshold, ie, the lower limit of |
| | the 95% CI is greater than 36%. |
| | Secondary Outcome Measures: |
| | Cohorts 1, 2, 3B and 4: Duration of Response (DoR) [Time Frame: 2 years |
| | and 2 months] |
| | DoR is defined as the time from the date of first documented response (PR or CR) |
| | until the date of documented progression or death, whichever comes first, for |
| | participants who have PR or CR. |
| | Cohorts 1, 2, 3B and 4: Clinical Benefit Rate (CBR) [Time Frame: 2 years |
| | and 2 months] |
| | CBR is defined as the percentage of participants achieving a confirmed complete or |
| | partial response, or durable stable disease (duration of at least 6 weeks) as defined by |
| | RECIST version 1.1. |
| | Cohorts 1, 2, 3B and 4: Progression-free Survival (PFS) [Time Frame: 2 |
| | years and 2 months] |
| | PFS is defined as the time from the first administration of study treatment until the |
| | date of objective disease progression or death, whichever comes first, based on |
| | investigator assessment using RECIST version 1.1. |
| | Cohorts 1, 2, 3B and 4: Overall Survival (OS) [Time Frame: 2 years and 2 |
| | months] |
| | OS is defined as the time from the first administration of study treatment until the |
| | date of death due to any cause. |
| | Cohorts 1, 2, 3B and 4: Number of Participants With Treatment-emergent |
| | Adverse Events (TEAEs) as a Measure of Severity [Time Frame: 2 years and 1 |
| | month] |
| | An AE is any untoward medical occurrence in a participant participating in a clinical |
| | study that does not necessarily have a causal relationship with the pharmaceutical/ |
| | biological agent under study. TEAEs are defined as AEs with onset or worsening on |
| | or after date of first dose of study treatment. Severity of TEAEs will be graded |
| | according to the National Cancer Institute Common Terminology Criteria for |
| | Adverse Events (NCI-CTCAE) version 5.0. Severity scale ranges from Grade 1 |
| | (Mild) to Grade 5 (death). Grade 1 = mild, Grade 2 = moderate, Grade 3 = severe, |
| | Grade 4 = life-threatening and Grade 5 = death related to adverse events. |
| | Cohort 1 and 4: Maximum Observed Serum Concentration (Cmax) of |
| | Amivantamab [Time Frame: Predose up to 168 hours post dose on Day 1] |
| | Cmax is defined as maximum observed serum concentration of amivantamab. |
| | Cohort 1 and 4: Time to Maximum Observed Serum Concentration (Tmax) |
| | of Amivantamab [Time Frame: Predose up to 168 hours post dose on Day 1] |
| | Tmax is defined as time to maximum observed serum concentration of |
| | amivantamab. |
| | Cohort 1 and 4: Area Under the Serum Concentration Curve Verses Time |
| | Curve From Time tl to t2 (AUC[t1-t2]) of Amivantamab [Time Frame: Predose up |
| | to 168 hours post dose on Day 1] |
| | AUC(t1-t2) is defined as area under the serum concentration versus time curve from |
| | time tl to time t2 of amivantamab. |
| | Cohort 1 and 4: Area Under the Curve From Time Zero to tau (AUC[0-tau]) |
| | of Amivantamab [Time Frame: Predose up to 168 hours post dose on Day 1] |
| | AUC(0-tau) is defined as area under the curve from time 0 to tau hours of |
| | amivantamab. |
| | Cohort 1 and 4: Trough Serum Concentration (C_{trough}) of Amivantamab |
| | [Time Frame: Predose up to 168 hours post dose on Day 1] |
| | Ctrough is defined as the serum concentration of amivantamab. |
| | Cohort 1 and 4: Accumulation Ratio (R) of Amivantamab [Time Frame: |
| | Predose up to 168 hours post dose on Day 1] |
| | Accumulation ratio (R) is calculated as area under the serum concentration-time |
| | curve from time zero to 168 hours (AUC[0-168]) value at Cycle 2 Day 1 dose |
| | divided by AUC(0-168) value after Cycle 1 Day 1 amivantamab dose. |
| | Cohort 5: |
| | To further assess anti-tumor activity of amivantamab in addition to |
| | pembrolizumab + carboplatin in participants with R/M HNSCC who are treatment- |
| | naive in the R/M setting. The Endpoints include: Duration of Response (DoR), |
| | Clinical Benefit Rate (CBR), Progression-free Survival (PFS), and Overall Survival |
| | (OS). |
| | To further characterize safety and tolerability of amivantamab in addition to |
| | pembrolizumab + carboplatin in participants with R/M HNSCC who are treatment- |
| | naive in the R/M setting, by evaluating incidence and severity of TEAEs. |

### Example 2. OrigAMI-4 Clinical Trial: Interim Results

Thirty-one patients were enrolled. Twenty-three patients had disease evaluation. Patients in amivantamab monotherapy cohort (Cohort 1) received subcutaneous injection of amivantamab monotherapy at a dose 1600 milligrams (mg) (2240 mg, if body weight >=80 kilograms [kg]) on Cycle 1 Day 1 and 2400 mg (3360 mg, if body weight >=80 kg) once every week (Q1W) for the remainder of Cycle 1 (Days 8 and 15), and every 3 weeks (Q3W) from Cycle 2 onwards.

Key eligibility criteria included (1) histologically or cytologically confirmed recurrent or metastatic HNSCC considered uncurable by local therapies, (2) prior platinum-based chemotherapy and PD-1/PD-L1 inhibitor, and (3) no prior anti-EGFR therapy or >2 prior lines of systemic therapy.

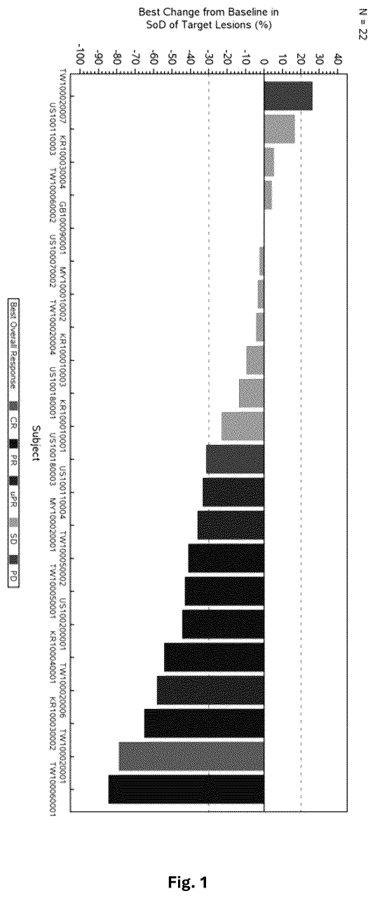

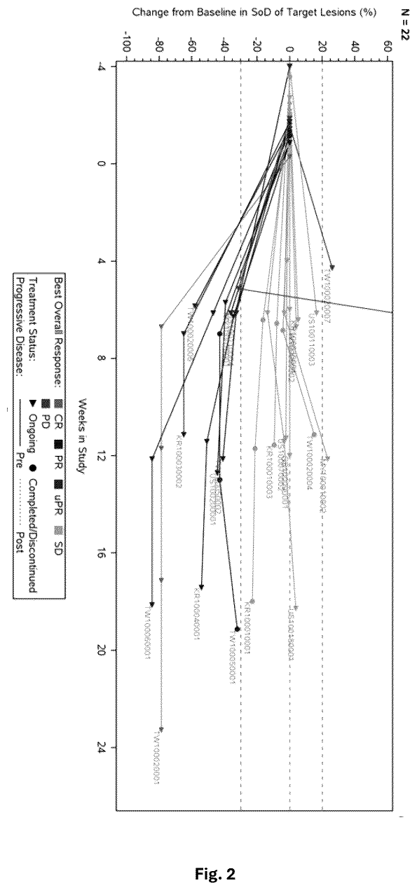

Baseline characteristics are shown in Table 2. Total number of treatment cycles received by patients, is shown in Table 3. Efficacy data are shown in Table 4, FIG. 1 and FIG. 2.

TABLE 2
Baseline characteristics.
Baseline Characteristics (N = 31)

| | |
|---|---|
| Age, median | 67 |
| Sex, n (%) | |
| Female | 9 (29) |
| Male | 22 (71) |
| ECOG PS, n (%) | |
| 0 | 12 (38.7) |
| 1 | 19 (61.3) |
| Race, n (%) | |
| Asian | 16 (51.6) |
| White | 13 (41.9) |
| Not reported | 2 (6.5) |
| Primary tumor location, n (%) | |
| Oral Cavity | 15 (48.4) |
| Oropharynx | 4 (12.9) |
| Hypopharynx | 2 (6.5) |
| Larynx | 10 (32.2) |
| Location of metastasis, n (%) | |
| Lymph node | 12 (46.2) |
| Lung | 16 (61.5) |
| Other | 9 (34.6) |

TABLE 3
Total number of cycles.

| Total number of cycles | n (%) |
|---|---|
| ≥1 cycle | 31 (100.0%) |
| ≥2 cycles | 28 (90.3%) |
| ≥3 cycles | 22 (71.0%) |
| ≥4 cycles | 14 (45.2%) |
| ≥5 cycles | 11 (35.5%) |
| ≥6 cycles | 7 (22.6%) |
| ≥7 cycles | 3 (9.7%) |
| ≥8 cycles | 1 (3.2%) |
| ≥9 cycles | 1 (3.2%) |

TABLE 4
Efficacy.
Cohort 1

| | |
|---|---|
| ORR | 48% |
| CR | 1 |
| PR | 10 |
| SD | 10 |
| PD | 2 |
| Disease evaluation, n | 23 |
| Ongoing treatment, n (%) | 26 (84%) |

ORR-overall response rate;
CR-complete response;
PR-partial response;
SD-stable disease;
PD-progressive disease.

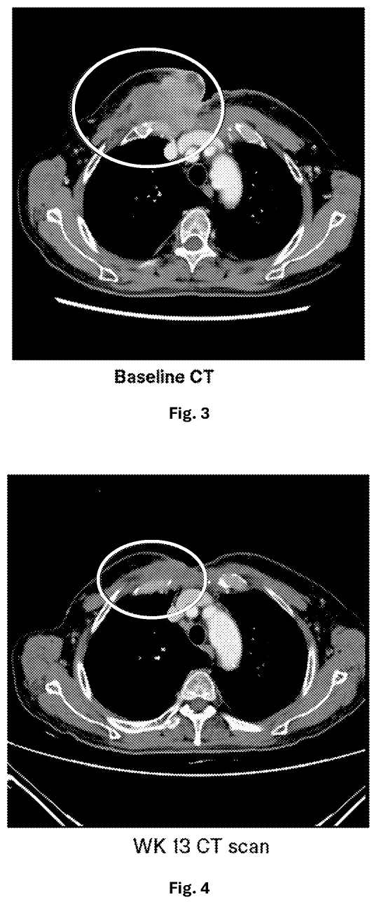

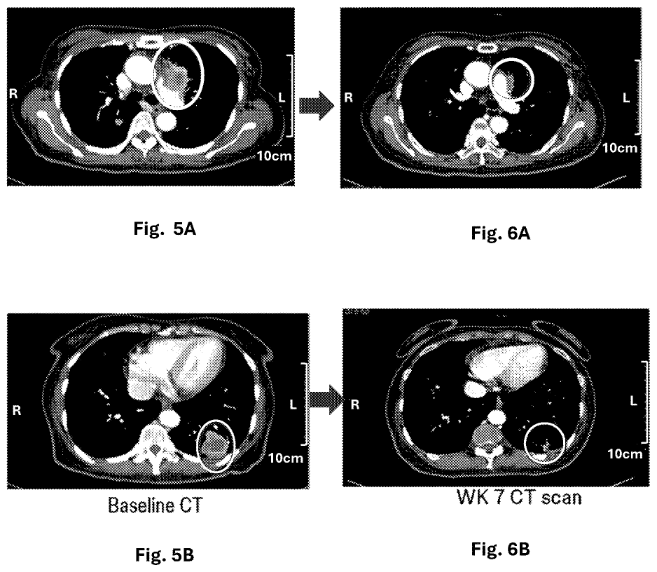

Representative patients from Cohort 1 are described below.
Patient 1. Sixty-one years old male diagnosed with metastatic oral cavity cancer. Prior treatments included chemoradiation, surgery, palliative radiation, resection for sternal metastasis, docetaxel+cisplatin, nivolumab. Baseline computer tomography (CT) scan is shown on FIG. 3. CT scan performed 13 weeks after the beginning of treatment showed 51% reduction of sternal mass in response to amivantamab treatment, see FIG. 4.
Patient 2. Forty-eight years old female diagnosed with metastatic oral cavity cancer. Prior treatments included surgery, cisplatin/radiation ×2, pembrolizumab. Baseline CT scans are shown on FIG. 5A and FIG. 5B. CT scans performed 7 weeks after the beginning of treatment showed dramatic reduction of both sternal masses in response to amivantamab treatment, see FIG. 6A and FIG. 6B.
Patient 3. Sixty-nine years old male diagnosed with oral cavity cancer. Prior treatments included surgery, radiation, carboplatin/paclitaxel/pembrolizumab, nivolumab/ipilimumab/methotrexate. Progression of the disease resulted in retraction from the tumor in right cheek and lip edema. Upon starting the treatment with amivantamab, quick response after 1 dose of amivantamab was observed, resulting in a formation of fistula due to rapid elimination of tumor cells. 85% response after 1 cycle of amivantamab was observed.

### Example 3. a Phase 3 Open-Label Study of Amivantamab in Addition to Carboplatin and Pembrolizumab, in Comparison with Standard of Care Platinum/Pembrolizumab/5-FU, in Participants with Treatment-Naïve Recurrent/Metastatic Head and Neck Squamous Cell Carcinoma

This is a randomized, multicenter Ph3 study of amivantamab (SC) in addition to pembrolizumab and carboplatin therapy in comparison to pembrolizumab, platinum therapy (carboplatin/cisplatin), and 5-FU in participants with recurrent and/or metastatic (R/M) HNSCC, who are treatment-naïve in the R/M setting, with CPS≥1. Participants must be treatment-naïve in the R/M setting. Participants must have local testing results demonstrating a Combined Positive Score (CPS) ≥1. Participants must not have received prior anti-EGFR therapy. Participants with primary oropharyngeal tumors must have HPV testing results (p16 test or high-risk HPV ISH in tissue) available.

Objectives and Endpoints

| | |
|---|---|
| Objectives | Endpoints |

In participants treated with amivantamab + pembrolizumab + carboplatin vs.
5-FU + pembrolizumab + platinum (carboplatin/cisplatin) therapy
Primary

| | |
|---|---|
| To compare anti-tumor activity | OS |
| | ORR as assessed by blinded independent |
| | central review (BICR) |

Secondary

| | |
|---|---|
| To further assess measures of clinical | PFS (using RECIST vl.1), as assessed by |
| benefit | BICR |
| | DoR, as assessed by BICR |
| | ORR as assessed by the investigator |
| To assess safety and tolerability | Incidence of TEAE |
| To assess disease symptoms, HRQOL, and | Proportion of patients with improved or stable |
| treatment tolerability | symptoms relative to baseline, as measured by |
| | the EORTC QLQ-HN43 and symptom scales |
| | of the EORTC QLQ-C30 |
| | Change from baseline in functioning and |
| | overall HRQOL, as measured by functioning |
| | and global health scales of the EORTC QLQ- |
| | C30 |
| | Differences between treatment groups in the |
| | CTSQ Feelings About Side Effects scale scores |

Exploratory

| | |
|---|---|
| To further assess measures of clinical | PFS (using RECIST vl.1), assessed by |
| benefit | investigator |
| | DoR, assessed by investigator |
| For amivantamab-treated participants only: | Serum amivantamab concentrations and serum |
| To explore the relationship between PK or | anti-amivantamab antibodies |
| immunogenicity and selected endpoints in | |
| amivantamab-treated participants (including | |
| but not limited to efficacy and safety) | |
| To explore the mechanisms of action of | |
| study arms | |
| To explore biomarkers predictive of | |
| clinical response and resistance to all | |
| study agents | |
| To further assess HRQOL | Change from baseline in HRQOL, as measured |
| | by EQ-5D-5L utility score and EQ-VAS |
| | Disease control rate |
| | Time to treatment failure |

Inclusion Criteria
Age

| | |
|---|---|
| 1. | Participant must be ≥18 years of age (or the legal age of majority in the jurisdiction in which |
| | the study is taking place, whichever is greater). |

Type of Participant and Disease Characteristic(s)

| | |
|---|---|
| 2. | Participant must have an ECOG performance status of 0 or 1. |
| 3. | Participant must have histologically or cytologically confirmed R/M HNSCC that is |
| | considered incurable by local therapies. |
| | The eligible primary tumor locations are the oral cavity, oropharynx, hypopharynx, or |
| | larynx. |
| | Participants must not have a primary tumor site of nasopharynx (any histology). |
| | Participants must have documented local testing results per local regulations using a |
| | 22C3 antibody assay for PD-L1 status demonstrating a Combined Positive Score |
| | (CPS) ≥1 within 6 months of C1D1. Tissue used for CPS testing should be obtained |
| | after diagnosis of R/M disease. |
| | HPV status must be known for patients with primary tumor location in oropharynx via |
| | p16 test, HPV DNA test, or high-risk HPV ISH. Any known p16, HPV DNA, or high- |
| | risk HPV ISH status of tumor must be negative. |
| 4. | Participant must be treatment-naive for systemic therapy in the R/M setting. Systemic therapy |
| | which was completed more than 6 months prior to signing consent, if given as part of treatment |
| | for locally advanced disease with curative intent, is allowed. Participant must not have had |
| | disease progression within 6 months of completion of curative systemic therapy for locally |
| | advanced disease. Participant must not have received any prior anti-EGFR therapy and anti- |
| | MET therapy. Participant may have received anti-PD-1/PD-L1/PD-L2 agents if given >12 |
| | months prior to enrollment. |
| 5. | Participant must have measurable disease according to RECIST v1.1. If only one measurable |
| | lesion exists, it may be used for the screening biopsy as long as baseline tumor assessment scans |
| | are performed ≥7 days after the biopsy. Tumor lesions situated in a previously irradiated area |
| | are considered measurable if progression has been demonstrated in such lesions. |
| 6. | Participants must consent to a screening biopsy or provide archival tissue sample per protocol- |
| | defined specifications. Participants must have a screening biopsy within 28 days from C1D1 or |
| | archival tissue that was obtained within 6 months of diagnosis of R/M disease. |

Sex and Contraceptive/Barrier Requirements

| | |
|---|---|
| 7. | While on study treatment and for 6 months after the last dose of study treatment, a participant |
| | must: |
| | a. Not breastfeed or be pregnant. |
| | a. Not donate gametes (ie, eggs or sperm) or freeze for future use for the purposes of assisted |
| | reproduction. |
| | b. Wear an external condom. |
| | c. If of childbearing potential, participant must: |
| | have a negative highly sensitive (eg, beta-human chorionic gonadotropin [ß- |
| | hCG]) serum pregnancy test at screening and within 72 hours of the first dose of |
| | study treatment and must agree to further pregnancy tests. |
| | practice at least 1 highly effective method of contraception; if oral contraceptives |
| | are used, a barrier method of contraception must also be used. |
| | d. If a partner of a participant capable of producing sperm is of childbearing potential, the |
| | partner must practice a highly effective method of contraception unless the participant is |
| | vasectomized. A participant who is vasectomized must still use a condom, but the partner |
| | is not required to use contraception. |
| | Participants should consider preservation of gametes prior to study treatment as anti-cancer |
| | treatments may impair fertility. |

### Exclusion Criteria

Any potential participant who meets any of the following criteria will be excluded from participating in the study:
1. Has an uncontrolled illness, including but not limited to the following:
   a. Diabetes.
   b. Ongoing or active infection (includes infection requiring treatment with antimicrobial therapy [participants will be required to complete antibiotics 1 week prior to starting study treatment]) or diagnosed or suspected viral infection.
   c. Active bleeding diathesis (including active bleeding from primary tumor, or recent bleeding within 2 weeks prior to first administration of study treatment requiring medical or surgical intervention).
   d. Impaired oxygenation requiring continuous oxygen supplementation.
   e. Psychiatric illness/social situation that would limit compliance with study requirements.
   f. Active autoimmune disease that has required systemic treatment in past 2 years (ie, with use of disease modifying agents, corticosteroids, or immunosuppressive drugs). Replacement therapy (eg, thyroxine, insulin, or physiologic corticosteroid replacement therapy for adrenal or pituitary insufficiency) is not considered a form of systemic treatment.
   g. Participant has had an allogeneic tissue/solid organ transplant.
2. Has untreated brain metastases or history of known presence of leptomeningeal disease. Notes: Participants with definitively, locally treated metastases that are clinically stable and asymptomatic for at least 4 weeks and who are off or receiving low-dose corticosteroid treatment (≤10 mg prednisone or equivalent) for at least 4 weeks prior to study treatment are eligible, provided they have not used steroids for at least 7 days prior to study treatment.
3. Has a history of (non-infectious) ILD/pneumonitis/pulmonary fibrosis, has current ILD/pneumonitis, or where suspected ILD/pneumonitis/pulmonary fibrosis cannot be ruled out by imaging at screening.
3. Has a history of clinically significant cardiovascular disease.

### Study Design and Drug Administration

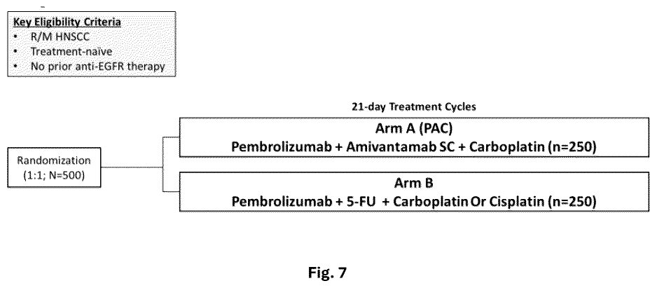

Study design is shown in FIG. 7. Description of treatments is shown in Table 5.

TABLE 5
Description of Treatments.

| | Arm A: | Arm B: |
|---|---|---|
| Group/Arm | Pembrolizumab + Amivantamab + | Pembrolizumab + 5-FU + |
| Name | Carboplatin (PAC) | Carboplatin/ Cisplatin |
| Treatment | Pembrolizumab | Pembrolizumab |
| Name | Amivantamab | 5-FU |
| | Carboplatin | Carboplatin/Cisplatin |
| Treatment | Pembrolizumab → Carboplatin → | Pembrolizumab → Carboplatin/ |
| Order | Amivantamab | Cisplatin → 5-FU |
| Type | Drug | Drug |
| Dosage | Pembrolizumab IV 200 mg Q3W | Pembrolizumab IV 200 mg Q3W |
| Level(s) | Amivantamab SC 1,600 mg (2,240 mg | 5-FU IV 1000 mg/m2 per day for 4 days |
| | ≥80 kg) on C1D1; and 2,400/3,360 mg | Q3W |
| | on C1D8 and C1D15 and Q3W from | Carboplatin IV AUC 5 mg/mL · min Q3W |
| | Cycle 2 onwards | Cisplatin 100 mg/m2 Q3W |
| | Carboplatin IV AUC 5 mg/mL · min | |
| | Q3W | |
| Route of | Pembrolizumab-Intravenous infusion | Pembrolizumab-Intravenous infusion |
| Administration | Amivantamab-Subcutaneous injection | 5-FU-Intravenous infusion |
| | Carboplatin-Intravenous infusion | Carboplatin-Intravenous infusion |
| | | Cisplatin-Intravenous infusion |
| Use | Experimental | Active Comparator |
| Investigational | Amivantamab-Yes | 5-FU-Yes |
| Medicinal | Pembrolizumab-Yes | Pembrolizumab-Yes |
| Product (IMP) | Carboplatin-Yes | Carboplatin-Yes |
| | | Cisplatin-Yes |
| Non- | — | — |
| Investigational | | |
| Medicinal | | |
| Product/ | | |
| Auxiliary | | |
| Medicinal | | |
| Product | | |
| (NIMP/AxMP) | | |

### Arm A Dosing: Pembrolizumab+Amivantamab+Carboplatin

Amivantamab will be dosed weekly during Cycle 1 and Q3W starting on Cycle 2 Day 1. pembrolizumab and carboplatin will be dosed Q3W starting from Cycle 1. The order of dosing combination agents will be: pembrolizumab, carboplatin, followed by amivantamab. Table 6 provides dosing information for the experimental Arm A.

Amivantamab will be dosed weekly during Cycle 1 and Q3W starting on C2D1. The amivantamab dosage will be based on the participant's baseline BW.

Pembrolizumab will be dosed Q3W starting from Cycle 1 onwards. Carboplatin will be dosed starting from Cycle 1 Q3W for a maximum of 6 cycles. The order of dosing combination agents in Arm A will be: pembrolizumab, carboplatin, followed by amivantamab.

Maximum duration of treatment for pembrolizumab and amivantamab in experimental arm is 24 months.

TABLE 6
Arm A: Pembrolizumab + Amivantamab + Carboplatin (PAC)

| 3-week | | | Amivantamab | Amivantamab |
|---|---|---|---|---|
| cycle | Pembrolizumabª | Carboplatin^{b} | BW <80 kg^{c} | BW ≥80 kg^{c} |
| Cycle 1 | 200 mg | AUC 5 | 1600 mg | 2240 mg |
| Day 1 | | | | |
| Cycle 1 | | | 2400 mg | 3360 mg |
| Day 8, | | | | |
| Cycle 1 | | | | |
| Day 15 | | | | |
| Cycle 2 | 200 mg | AUC 5 | 2400 mg | 3360 mg |
| Day 1 | | | | |
| onwards, | | | | |
| Q3W | | | | |

For Day 1 of each cycle, pembrolizumab should be administered first, followed by carboplatin (timing per the local guidelines and SmPC regulatory labeling) and amivantamab SC last. Amivantamab should be administered at least 1 hour after carboplatin infusion.
^{a}Pembrolizumab will be administered over a 30-minute infusion time per local product label/site practice. Pembrolizumab should not be administered as an IV push or bolus injection. Every effort should be made to target infusion timing to be as close to 30 minutes (-5 to +10 minutes) as possible.
^{b}Carboplatin will be administered for a target AUC of 5 mg/mL/min as an IV infusion on Cycle Day 1, Q3W. Pursuant to the CTEP Information Letter Regarding the AUC-Based Dosing of Carboplatin, the max carboplatin dose should not exceed the target AUC (mg*min/mL)*150 mL/min, but it may be less. The glomerular filtration rate (GFR) used in the Calvert formula to AUC-based dosing should not exceed 125 mL/min. For this trial, the max dose cannot exceed a total dose of 750 mg. Alterations in renal function may require a recalculation of dose. Administer over 15 to 60-min infusion time per local product label/site practice (Maximum 6 cycles permitted).
^{c}Amivantamab SC dosing will occur over three days for Cycle 1 (C1D1, C1D8 and C1D15) and only on D1 from Cycle 2 onwards. On C1D1, participants will be observed for 4 hours on site after amivantamab injection.

### Arm B Dosing: Pembrolizumab+5-FU+Platinum (Carboplatin/Cisplatin)

Arm B will follow the dosing approved from the KEYNOTE-048 study as outlined in Table 7 (Burtness 2019). The sequence of drug administration will be: pembrolizumab, followed by carboplatin/cisplatin, then 5-FU infusion. Pembrolizumab, carboplatin/cisplatin, and 5-FU will be dosed every three weeks starting on Cycle 1 Day 1. Carboplatin/cisplatin and 5-FU will be dosed for a maximum of 6 cycles. The maximum duration of treatment for pembrolizumab in comparator arm is 24 months.

The treatment with either carboplatin or cisplatin will be chosen prior to randomization. If a patient initially receives cisplatin, the patient may be switched from cisplatin to carboplatin during the course of the study if toxicities occur. If cisplatin dose was modified prior to switching, the subject may start a carboplatin dose of AUC 5 and will be eligible to receive an additional 2 dose modifications of carboplatin.

For 5-FU-related AEs, patients with intolerable or persistent Grade 2 drug-related AEs may hold drug. Permanently discontinue drug for persistent Grade 2 adverse reactions for which treatment has been held for 12 weeks, and grade has not recovered to 0 or 1 in severity. With lab-related AEs still at Grade 2 after 12 weeks, subject may continue on the trial if asymptomatic and controlled.

TABLE 7
Arm B: Pembrolizumab + 5-FU + Carboplatin/Cisplatin

| 3-week | | | | |
|---|---|---|---|---|
| cycle | Pembrolizumab^{a} | Carboplatin^{b} | Cisplatin^{c} | 5-FU^{d} |
| Cycle 1-6, | 200 mg | AUC 5 | 100 mg/m² | 1000 mg/m² |
| Day 1ª | | | | |
| Cycle 1-6, | — | — | — | 1000 mg/m² |
| Days 2, 3, | | | | |
| and 4 | | | | |
| Cycle 7 | 200 mg | — | — | |
| onwards, | | | | |
| Day 1 | | | | |

For Day 1 of each cycle, pembrolizumab will be administered first, followed by carboplatin and then 5-FU infusion. The choice of either carboplatin or cisplatin will be made before randomization.
^{a}Pembrolizumab will be administered at 200 mg over a 30-minute infusion time per local product label/site practice. Pembrolizumab should not be administered as an IV push or bolus injection. Every effort should be made to target infusion timing as close to 30 minutes (−5 to +10 minutes) as possible.
^{b}Carboplatin will be administered for a target AUC of 5 mg/mL/min as an IV infusion on Cycle Day 1 of a 3-week cycle. Pursuant to the CTEP Information Letter Regarding the AUC-Based Dosing of Carboplatin, the maximum carboplatin dose should not exceed the target AUC (mg*min/mL)*150 mL/min, but it may be less. The glomerular filtration rate (GFR) used in the Calvert formula to AUC- based dosing should not exceed 125 mL/min. For this trial, the maximum dose of carboplatin cannot exceed a total dose of 750 mg. Alterations in renal function may require a recalculation of the carboplatin dose. Carboplatin will be administered over a 15 to 60-minute infusion time per local product label/site practice (maximum 6 cycles permitted).
^{c}Cisplatin will be administered at 100 mg/m² using an infusion duration of 60 minutes (or infusion duration according to local practice) on D1 of each cycle (maximum 6 cycles are permitted).
^{d}5-FU will be administered at a dose of 1000 mg per square meter of body surface area per day as a continuous infusion for four days on Cycle Days 1-4 of a 3-week cycle (maximum 6 cycles are permitted).

### Example 4. Efficacy

Example patient responses treated in OrigAMI-4 cohort 5 with a combination of amivantamab, pembrolizumab, and carboplatin, are described below.
Patient 1: Sixty-nine year old male diagnosed with hypopharyngeal cancer. Prior treatments included chemotherapy and radiation therapy. The patient had a recurrence with a large pharyngeal mass. Upon starting treatment with the carboplatin/amivantamab/pembrolizumab combination, the patient had a complete response (CR) at the time of first disease evaluation.
Patient 2: Seventy year old male diagnosed with oral cavity cancer. Prior treatments included surgery and radiation therapy. The patient had a recurrence in the neck with two large masses. Upon starting treatment with the carboplatin/amivantamab/pembrolizumab combination, the patient had 72% shrinkage of tumors at the time of first disease evaluation, and a CR at the time of second disease evaluation.

1. A method of treating head and neck cancer in a subject in need thereof, comprising administering to the subject a combination therapy, wherein the combination therapy comprises
a therapeutically effective amount of an anti-epidermal growth factor receptor (EGFR)/hepatocyte growth factor receptor (c-Met) antibody;
a therapeutically effective amount of pembrolizumab; and
a therapeutically effective amount of carboplatin.

2. The method of claim 1, wherein the EGFR/c-Met antibody comprises:
a) a first domain that specifically binds EGFR, comprising heavy chain complementarity determining region 1 (HCDR1), HCDR2, HCDR3, light chain complementarity determining region 1 (LCDR1), LCDR2 and LCDR3 amino acid sequences of SEQ ID NOs: 1, 2, 3, 4, 5 and 6, respectively; and
b) a second domain that specifically binds c-Met, comprising HCDR1, HCDR2, HCDR3, LCDR1, LCDR2 and LCDR3 amino acid sequences of SEQ ID NOs: 7, 8, 9, 10, 11 and 12, respectively.

3. The method of claim 2, wherein the first domain comprises a heavy chain variable region (VH) of SEQ ID NO:13 and a light chain variable region (VL) of SEQ ID NO:14, and the second domain comprises a VH of SEQ ID NO: 15 and a VL of SEQ ID NO: 16.

4. The method of claim 1, wherein the antibody is of the IgG1 isotype.

5. The method of claim 1, wherein the antibody comprises a first heavy chain (HC1) of SEQ ID NO:17, a first light chain (LC1) of SEQ ID NO:18, a second heavy chain (HC2) of SEQ ID NO:19 and a second light chain (LC2) of SEQ ID NO:20.

6. The method of claim 1, wherein the antibody is administered at a dose of about 1,600 mg to about 3,360 mg.

7. The method of claim 6, wherein the antibody is administered at a dose of about 1,600 mg, about 2,240 mg, about 2,400 mg, or about 3,360 mg.

8. The method of claim 7, wherein the antibody is administered at a dose of about 1,600 mg.

9. The method of claim 7, wherein the antibody is administered at a dose of about 2,240 mg.

10. The method of claim 7, wherein the antibody is administered at a dose of about 2,400 mg.

11. The method of claim 7, wherein the antibody is administered at a dose of about 3,360 mg.

12. The method of claim 6, wherein the antibody is administered once a week or once every three weeks.

13. The method of claim 12, wherein the antibody is administered once weekly for the first 3 weeks and once every 3 weeks thereafter.

14. The method of claim 1, wherein the antibody is administered at a dose of about 1,600 mg (about 2240 mg, if body weight is equal or larger than 80 kg) on Cycle 1 Day 1 and about 2400 mg (about 3360 mg, if body weight is equal or larger than 80 kg) once every week (Q1W) for the remainder of Cycle 1 (Days 8 and 15), and once every 3 weeks (Q3W) from Cycle 2 onwards, wherein each Cycle is 21 days.

15. The method of claim 1, wherein the antibody is administered at a dose of about 1,600 mg on Cycle 1 Day 1 and about 2400 mg once every week (Q1W) for the remainder of Cycle 1 (Days 8 and 15), and once every 3 weeks (Q3W) from Cycle 2 onwards, wherein each Cycle is 21 days, if body weight is less than 80 kg.

16. The method of claim 1, wherein the antibody is administered at a dose of about 2240 mg on Cycle 1 Day 1 and about 3360 mg once every week (Q1W) for the remainder of Cycle 1 (Days 8 and 15), and once every 3 weeks (Q3W) from Cycle 2 onwards, wherein each Cycle is 21 days, if body weight is equal or larger than 80 kg.

17. The method of claim 1, wherein pembrolizumab is administered at a dose of about 200 mg once every three weeks.

18. The method of claim 1, wherein carboplatin is administered at a dose of about AUC 5 mg/mL·min once every three weeks.

19. The method of claim 1, wherein the head and neck cancer is a head and neck squamous cell carcinoma (HNSCC).

20. The method of claim 19, wherein the HNSCC is cancer of oropharynx, oral cavity, hypopharynx, or larynx.

21. The method of claim 20, where the HNSCC is HPV unrelated.

22. The method of claim 20, wherein the HNSCC is HPV negative.

23. The method of claim 20, wherein the HNSCC is HPV positive.

24. The method of claim 20, wherein the HNSCC HPV infection status is unknown.

25. The method of any one of claim 1-24, wherein the subject is treatment naïve

26. The method of claim 1, wherein the subject is treatment-naïve in a recurrent/metastatic head and neck cancer setting.

27. A method of treating recurrent or metastatic head and neck squamous cell carcinoma in a subject in need thereof comprising administering a therapeutically effective amount of the anti-EGFR/anti-cMet antibody (e.g., amivantamab), pembrolizumab, and carboplatin, wherein the subject is treatment-naïve in a recurrent/metastatic head and neck cancer setting, wherein the anti-EGFR/anti-cMet antibody is administered in 21-day cycles (i) in an amount of 1,600 mg if the subject has a body weight of less than 80 kg (or an amount of 2,240 mg if the subject has a body weight of ≥80 kg) on Cycle 1 Day 1, and then (ii) in an amount of 2,400 mg if the subject has a body weight of less than 80 kg (or an amount of 3,360 mg if the subject has a body weight of ≥80 kg) once weekly (Q1W) for the remainder of Cycle 1 (Days 8 and 15) and once every 3 weeks (Q3W) from Cycle 2 onwards, and wherein the pembrolizumab is administered at a dose of 200 mg Q3W (on Day 1 of each 21-day cycle) starting in Cycle 1 (e.g., as an intravenous injection), and wherein the carboplatin is administered at a dose of AUC 5 mg/mL·min Q3W (on Day 1 of each 21-day cycle) starting in Cycle 1 (e.g., as an intravenous injection).

28. The method of claim 1, wherein the anti-EGFR/anti-cMet antibody is administered subcutaneously.

29. The method of claim 19, wherein the HNSCC is a cancer of the oropharynx that is HPV negative.

30. The method of claim 27, wherein the head and neck cancer is a head and neck squamous cell carcinoma (HNSCC).

31. The method of claim 28, wherein the HNSCC is cancer of oropharynx, oral cavity, hypopharynx, or larynx.

32. The method of claim 28, wherein the HNSCC is a cancer of the oropharynx that is HPV negative.


SEQUENCE LISTING (42 sequences)

SEQ ID NO: 1 - protein, 5 aa, Homo sapiens
TYGMH

SEQ ID NO: 2 - protein, 17 aa, Homo sapiens
VIWDDGSYKYYGDSVKG

SEQ ID NO: 3 - protein, 16 aa, Homo sapiens
DGITMVRGVMKDYFDY

SEQ ID NO: 4 - protein, 11 aa, Homo sapiens
RASQDISSALV

SEQ ID NO: 5 - protein, 7 aa, Homo sapiens
DASSLES

SEQ ID NO: 6 - protein, 9 aa, Homo sapiens
QQFNSYPLT

SEQ ID NO: 7 - protein, 5 aa, Homo sapiens
SYGIS

SEQ ID NO: 8 - protein, 17 aa, Homo sapiens
WISAYNGYTNYAQKLQG

SEQ ID NO: 9 - protein, 10 aa, Homo sapiens
DLRGTNYFDY

SEQ ID NO: 10 - protein, 11 aa, Homo sapiens
RASQGISNWLA

SEQ ID NO: 11 - protein, 7 aa, Homo sapiens
AASSLLS

SEQ ID NO: 12 - protein, 9 aa, Homo sapiens
QQANSFPIT

SEQ ID NO: 13 - protein, 125 aa, Homo sapiens [sequence omitted]

SEQ ID NO: 14 - protein, 107 aa, Homo sapiens [sequence omitted]

SEQ ID NO: 15 - protein, 119 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 16 - protein, 107 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 17 - protein, 455 aa, Homo sapiens
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 18 - protein, 214 aa, Homo sapiens
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 20 - protein, 214 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, synthetic construct
NYYMY

SEQ ID NO: 22 - protein, 17 aa, synthetic construct
GINPSNGGTNFNEKFKN

SEQ ID NO: 23 - protein, 11 aa, synthetic construct
RDYRFDMGFDY

SEQ ID NO: 24 - protein, 15 aa, synthetic construct
RASKGVSTSGYSYLH

SEQ ID NO: 25 - protein, 7 aa, synthetic construct
LASYLES

SEQ ID NO: 26 - protein, 9 aa, synthetic construct
QHSRDLPLT

SEQ ID NO: 27 - protein, 120 aa, synthetic construct [sequence omitted]

SEQ ID NO: 28 - protein, 111 aa, synthetic construct [sequence omitted]

SEQ ID NO: 29 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 443 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 32 - protein, 447 aa, Homo sapiens [sequence omitted]

SEQ ID NO: 33 - protein, 446 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIF

SEQ ID NO: 34 - protein, 445 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 35 - protein, 444 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQ

SEQ ID NO: 36 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 37 - protein, 474 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 38 - protein, 509 aa, Homo sapiens [sequence omitted]

SEQ ID NO: 39 - protein, 474 aa, Homo sapiens
LNFRAPPVIPNVAFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSIWFLIISSVASL

SEQ ID NO: 40 - protein, 509 aa, Homo sapiens
MGVLQFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 41 - protein, 509 aa, Homo sapiens
MGVLKFKHIFFRSFVKSSGVSQIVFTFLLIPCCLTLNFRAPPVIPNAPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 42 - protein, 474 aa, Homo sapiens
LNFRAPPVIPNAPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL